(19) United States
(12) Patent Application Publication
(10) Pub. No.: US 2008/0194922 A1
(43) Pub. Date: Aug. 14, 2008

(54) POTENTIATION FOR MEDICAL THERAPIES

(76) Inventor: James F. Holden, Barrington Hills, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(21) Appl. No.: 11/863,946

(22) Filed: Sep. 28, 2007

Related U.S. Application Data
(63) Continuation-in-part of application No. 11/221,360, filed on Sep. 7, 2005.

(51) Int. Cl. A61B 5/00; A61B 10/00

(52) U.S. Cl. 600/300; 128/898

(57) ABSTRACT

This invention relates to providing medical treatment for human and non-human patients. In particular, the invention provides methods and computer program products which reduce clinical data complexity, aid in the identification of actions taken during the course of care which result in disproportionate therapeutic effects, and allow for the “potentiation” of therapy in an individual patient.

### CROSS-REFERENCE TO RELATED APPLICATIONS

This patent application is a continuation-in-part of copending U.S. patent application Ser. No. 11/221,360, filed on Sep. 7, 2005 and which is hereby incorporated by reference in its entirety.

### BACKGROUND OF THE INVENTION

The practice of medicine has gone far beyond its origins of merely making a patient comfortable while, hopefully, nature provided a cure. Various forms of intervention, sometimes helpful, sometimes not, have been tried over the millennia until today there is a vast quantity of diverse, complex accumulated knowledge that is beyond the capability of any one person to know. In extremely serious situations, even today, the goal is often to simply stabilize the condition of the patient. Once stabilized, the next goal is to render the disease chronic but manageable. The ultimate goal is a cure. It is unlikely that the same treatment can move a patient from acute and critical through chronic but manageable to cured. Thus, a need exists to coordinate accumulated wisdom with the patient condition's to effect the desired transitions. To do this also requires an integrated understanding of the patient's condition.

Treatment begins with a decision of whether to treat symptoms, the disease, or both. Then the physician decides whether to treat pharmacologically or surgically, or both. Each course has risks, benefits, and side effects. For many serious diseases such as cancer and infections, the treatment is complicated by interactions among agents that can damage healthy tissue and organs. Thus, when fighting disease, just as each person's condition is unique at some level of detail, each treatment is unique and it requires great skill on the part of a physician to define a treatment suited to each patient.

In the complex setting of modern medical care there may even be a problem in knowing whether one is helping or hurting a patient. One aspect of this problem is knowing what to monitor. For some therapies, the full range of side effects and interactions may not be known. Moreover, some side effects and interactions may result from a given patient's unique genetic make-up. Such patient-specific side effects and interactions may be extremely rare and not known to be correlated with a specific therapy.

Drug therapy is particularly characteristic of the complexity faced by physicians. The current approach to drug treatment is to give all patients with a given disease the same drug at an average dose, evaluate how it works, and then make adjustments as needed. But, there can be great heterogeneity in patient responses to any drug. For example, there are defects in the enzyme thiopurine methyltransferase which prevent patients from metabolizing the anti-cancer drug 6-mercaptopurine. Thus, one patient may need a “full” dose of 6-mercaptopurine; while another, who has a mutation in the gene, may need less than 10 percent of that dose.

Thus, genes play an important role one's susceptibility to disease and response to therapies including drugs. But, the complexity involved in using genes to determine therapy is illustrated by the vast number of genes, about 25,000, in each human being's complete gene complement. The current understanding of how 25,000 genes interact with each other is far from complete. Nonetheless, personalizing medical care to the individual patient is a necessity that can not wait for a perfect understanding of genes. Accordingly, to achieve the goal of personalizing care, there remains a need to reduce the complexity the physician faces and identify the actions which are correlated with most important changes occurring over time during the administration of a therapy to an individual patient.

The complexity that results from heterogeneity is dramatically evident in cancer, which is very diverse, complex, unpredictable, stochastic and evolutionary in nature. This genetic and epigenetic complexity causes cancer to appear “intelligent,” in terms of its ability to proliferate, invade, defeat apoptosis, develop innate alternative pathways when challenged, and more. As a result, cancer intelligently responds to a patient's changing condition and the therapy that is driving that change. Adding to this complexity is the indirect manner in which cancer operates, utilizing normal cellular functionality. Patients often do not die from cancer, but rather die from the effects of the body fighting the cancer, such as inflammation, lack of nutrition, opportunistic disease and other factors. It is this indirect and ever changing and adaptive nature of cancer that geometrically increases the complexity in curing cancer.

The complexity seen with gene-based medical care and cancer is also typical of more conventional areas of medicine. This is especially the case in acute care settings. For example, during labor the obstetrician must manage the health of both the baby and the mother. This requires that the obstetrician monitor fetal parameters including heart rate, blood gas levels, the size and position, presentation, and the position of the umbilical cord, while being alert for signs indicative of fetal distress such as meconium in the amniotic fluid. At the same, the obstetrician must be constantly aware of the maternal heart rate, blood pressure, pain level, placental intactness, frequency and nature of contractions, blood chemistries, blood counts, and pulmonary function. The management of labor is, thus, a dynamic process and includes the use of drugs which enhance or diminish uterine contractions, methods pain control, the management of the maternal blood volume, instrumental delivery, and cesarean section. The potential for catastrophic events such as uterine rupture or hemorrhage fatal to both the mother and baby are ever-present. Accordingly, to optimize acute care, there is a need for methods which simplify the complexity of the flood of data generated during labor and focus the obstetrician's attention on the components of therapy which correlate with the most important changes in the condition of the mother and baby.

Thus, the ability to readily identify which actions have had the greatest positive or negative effects on the patient would be of great value to the optimization of a therapy. Actions with disproportionate effects often impact points of “feedback” control in a therapeutic system. “Feedback” is a signal that is looped back to control a system within itself. This loop is called the “feedback loop.” Feedback may be “negative,” which reduces output; “positive,” which tends to increase output, or “bipolar,” which can either increase or decrease output. Human physiology frequently relies on feedback loops, for example, blood pressure and cell growth are controlled by a negative feedback loops. Since positive feedback loops enhance or amplify changes, if a positive feedback loop is involved in the response to a therapy, its stimulation should be emphasized. Conversely, the stimulation of a positive feedback loop that accentuates side effects should be avoided. Accordingly, there remains a need to readily identify which of the host of decisions made during the course of medical care have had disproportionate effects.

In view of the foregoing, it is therefore an object of the invention to provide a method for analyzing data to provide minimum dose for maximum therapeutic effect.

Another object of the invention is to provide a method for analyzing data to correlate treatment and symptoms.

A further object of the invention is to provide a method for treatment that can accommodate changes in treatment with changes in the condition of the patient.

Another object of the invention is to provide a method that focuses on the fewest number of elements in formulating a therapy.

A further object of the invention is to provide a method that allows time for the treatment to reach maximum effect at minimum dose.

Another object of the invention is to provide a method that combines elements to obtain a disproportionate, positive effect compared with linear or composite therapies.

A further object of the invention is to provide a method for analyzing data to provide minimum dose for maximum therapeutic effect, minimal side effects, and improved patient stability.

### BRIEF SUMMARY OF THE INVENTION

This invention relates to providing medical treatment for human and non-human patients. In particular, the invention provides methods and computer program products which reduce clinical data complexity, aid in the rapid identification of actions taken during the course of care which result in disproportionate effects, and, thus, allow for the “potentiation” of therapy in an individual patient.

Accordingly, the invention provides methods for potentiating a therapy in a patient comprising selecting a plurality of therapy elements and a plurality of correlation factors; collecting data relative to the therapy elements and the correlation factors at a plurality of time points in a timecourse; and analyzing the data. Data analysis is desirably done by calculating a difference between the therapy elements for each successive pair of time points and a difference between the correlation factors for each successive pair of time points; digitizing each of the calculated differences; calculating a product of the digitized difference for each therapy element from each successive pair of time points multiplied by the corresponding digitized difference for each correlation factor from each successive pair of time points; and calculating respective rolling averages of these products over the timecourse. One or more of the respective rolling averages are then displayed and the therapy is modified in accordance with the display.

The invention also provides methods for potentiating a therapy in a patient comprising selecting a plurality of therapy elements and a plurality of correlation factors; collecting data relative to the therapy elements and the correlation factors at a plurality of time points in a timecourse; and using an alternative approach to analyzing the data. Data analysis is done by calculating a difference between the therapy elements for each successive pair of time points and a difference between the correlation factors for each successive pair of time points; normalizing each of the calculated differences; calculating the product of the normalized difference for each therapy element for each successive pair of time points multiplied by the corresponding normalized difference for each correlation factor for each successive pair of time points; and calculating respective rolling averages of the products over the timecourse. One or more of the respective rolling averages are then displayed and the therapy is modified in accordance with the display.

In view of the data volume generated by and calculation intensity of the methods provided herein, the invention further provides computer program products comprising a computer-readable media having thereon computer-executable instructions for the performance of a method for potentiating a therapy in a patient, said method comprising the selecting a plurality of therapy elements; selecting a plurality of correlation factors; collecting data relative to the therapy elements and the correlation factors at a plurality of time points in a timecourse; storing the collected data in an relational database; and analyzing the data. Data analysis is done by calculating a difference between the therapy elements for each successive pair of time points and a difference between the correlation factors for each successive pair of time points; digitizing each of the calculated differences; calculating the product of the digitalized difference for each therapy element for each successive pair of time points multiplied by the digitalized difference for each correlation factor for each successive pair of time points; and calculating a rolling average of the products over the timecourse. The computer program products then display one or more of the rolling averages and provide a recommendation for a modification of the therapy in accordance with the display.

In other embodiments the invention provides computer program products comprising computer-readable media having thereon computer-executable instructions for the performance of a method for potentiating a therapy in a patient, said method comprising selecting a plurality of therapy elements; selecting a plurality of correlation factors; collecting data relative to the therapy elements and the correlation factors at a plurality of time points in a timecourse; storing the collected data in an relational database; and analyzing the data. Data analysis is accomplished by: calculating a difference between the therapy elements for each successive pair of time points and a difference between the correlation factors for each successive pair of time points; normalizing each of the calculated differences; calculating the product of the normalized difference for each therapy element for each successive pair of time points multiplied by the normalized difference for each correlation factor for each successive pair of time points; and calculating a rolling average of the products over the timecourse. The computer program products then display one or more of the rolling averages and provide a recommendation for a modification of the therapy in accordance with the display.

In preferred embodiments of the invention the method is repeated one or more times, either independently of or using an embodiment of the computer program products in accordance with the invention, while modifying one or more therapy elements on each repetition of the method. Such repetitions, done in accordance with the invention, include embodiments wherein the method is repeated until at least one predetermined outcome metric is satisfied.

In addition, the invention provides methods for potentiating a therapy in a patient using System Functions and computer program products comprising a computer-readable media having thereon computer-executable instructions for the performance of methods for potentiating a therapy using System Functions. Said methods for potentiating a therapy in a patient using System Functions comprise the steps of: selecting a plurality of therapy elements, selecting a plurality of correlation factors, collecting data relative to the therapy elements and the correlation factors at a plurality of time points in a timecourse; and analyzing the data. Data analysis in said methods using System Functions is done by: calculating a difference between the therapy elements for each successive pair of time points and a difference between the correlation factors for each successive pair of time points, processing the difference by applying a function from the group consisting of digitizing each of the calculated differences and normalizing each of the calculated differences, calculating a product of the processed difference for each therapy element from each successive pair of time points multiplied by the corresponding digitized or normalized difference for each correlation factor from each successive pair of time points, calculating respective rolling averages of the products over the timecourse, and using linear regression analysis to determine a System Function for each therapy element-correlation factor pair. The final steps of said methods using System Functions are: displaying one or more of the rolling averages and one or more of the Systems Functions and modifying the therapy in accordance with the displayed rolling averages and Systems Functions.

The invention also provides methods and computer program products for “potentiating” the treatment in groups of human and non-human patients suffering from the same condition.

### BRIEF DESCRIPTION OF THE SEVERAL VIEWS OF THE DRAWINGS

FIG. 1. depicts the four classes of protocols, within the two dimensions of Therapeutic Effect (TE) and Patient Stability (PS).

FIG. 2. is a flow chart of a process used to potentiate the therapy in accordance with the invention.

FIGS. 3-10. are data illustrating the invention.

FIG. 11. illustrates the operation of a computer program in accordance with a preferred embodiment of the invention.

FIG. 12. is a chart based upon the data from FIG. 1 through FIG. 8 using a rolling average of thirty days.

FIG. 13. is a chart based upon a subset of data and using a rolling average of eighteen days.

FIG. 14. is a chart illustrating the effect of averaging period.

FIG. 15. is a chart illustrating the effect of phase shift (time offset).

FIG. 16. is a flow chart of the process used to potentiate the therapy of multiple myeloma in cat.

FIG. 17 depicts the effect of time offset analysis on the normalized delta correlation function.

FIG. 18 depicts the effect of dexamethasome on food intake with no time offset.

FIG. 19 depicts the effect of dexamethasome on food intake with a 1 day offset.

FIG. 20 depicts the effect of dexamethasome on food intake with a 2 day offset.

FIG. 21 depicts hypothesis testing in accordance with the invention comparing three dexamethasome regimens' effect on food intake.

### DETAILED DESCRIPTION OF THE INVENTION

### I. Definitions

The invention provides methods of “potentiating” the therapy of a single patient or a group of patients with the same condition. By “potentiating a therapy” it is meant the creation of a therapeutic progression in the multimodal treatment of a patient with a serious condition which is based on the identification and exploitation of feedback points in the pathways producing responses to said multimodal treatment in that patient. Thus, potentiating the therapy results in the optimal outcome for the individual patient. In addition, the invention provides computer program products that carry out methods whereby the therapy of a single patient or a group of patients with the same condition is “potentiated.”

By “therapy elements” it is meant metrics of the care and treatment provided. Any suitable metrics of the care and treatment can be used as a therapy element including, for example the doses and/or frequencies of administration of chemotherapeutic agents, radiation therapies, antibiotics, antifungals, antivirals, hormones, immuno-modulators, vaccines, NSAIDs, opiates, analgesics, anaesthetics, antihypertensives, anticoagulants, oral hypoglycemics, statins, fibrates, neuroleptics, antidepressants, lithium, anixiolytics, herbal extracts, traditional medicines, and nutritional supplements, as well as the measurable characteristics of surgical procedures, psychotherapy, and the patient's diet.

Further, there can be “primary” therapy elements, which measure therapies directed at the disease process, and “secondary” therapy elements, which measure levels of supportive care. For example, in case of multiple myeloma, primary therapy elements include doses and mode of administration of thalidomide, dexamethasone, and Reishi (ganoderma lucidum) and the secondary therapy elements include the doses and mode of administration of nutritional supplements such as magnesium aspartate, potassium gluconate, ferrous gluconate, vitamin B complex, controlled exposure to natural sunlight (facilitating production of vitamin A in cats) and EPA/DHA (highly unsaturated fats in fish oil).

By “correlation factors” it is meant metrics that are indicative of the condition of the patient. Any suitable metric can be used as a correlation factor including, for example, blood values, disease state, side effects, appetite, waking weight, metabolism, balance/equilibrium, mental acumen, physical strength, endurance, dexterity, attitude, pain, blood values, vital signs, and side effects, e.g. peripheral neuropathy, lethargy, constipation, other physiologic parameters, patient performance status, patient symptoms, patient reported subjective factors, family reported subjective factors, complications, and patient care costs.

“Administration” is meant as a generic term for providing a treatment. For example, a drug may be administered orally, intravenously, or transdermally. The administration of a drug may be “modulated”, e.g. given one day and not another or given every four hours but only during a certain part of a day. Administration also relates to timing, e.g. time of day when a drug is administered.

In a “linear” therapy, a number of elements are applied sequentially, often as part of a trial-and-error effort. In the case of a severe infection, the elemental therapy may become a linear therapy when different antibiotics are administrated sequentially, until the most effective one is identified. This takes time and prolongs patient discomfort and recovery. In the case of cancer, a pattern of disease reduction, remission, and relapse often occurs, as with elemental therapy, with side effects that require additional therapies.

In a “composite” therapy, a number of similar and diverse, often complementary, elements are given that remain somewhat fixed during the course of therapy; e.g. penicillin and Lactobacillus acidophilus (to replace the bacteria naturally occurring in the intestine that may be killed by penicillin or other antibiotic). In the case of cancer, a pattern of disease reduction, remission, and relapse often occurs, as with elemental therapy and linear therapy, but to a lesser extent, as treatment failure rates are lower and disease progression is slower with fewer side effects due to less than maximum dosing.

By “timecourse” it is meant a complete study in which data is collected at specific times after the beginning of the study. The appropriate timecourse length depends on the patient's condition, the therapy factors controlled, and the correlation factors measured. Suitable timecourses include, for example, at least about 5 minutes, at least about 10 minutes, at least about 15 minutes, at least at least about 20 minutes, at least about 30 minutes, at least about 40 minutes, at least about 50 minutes, at least about 60 minutes; at least about 2 hours, at least about 3 hours, at least about 6 hours, at least about 8 hours, at least about 12 hours, at least about 24 hours; at least about 2 days, at least about 3 days, at least about 5 days, at least about 7 days, at least about 10 days, at least about 14 days, at least about 21 days, at least about 30 days, at least about 60 days, at least about 90 days, at least about 120 days, at least about 180 days, at least about 365 days, at least about 730 days, at least about 1,000 days, and for the live of the patient.

By “time point” it is meant the time after the beginning of the study at which a reading, observation or determination of a value for a therapy element or correlation factor is completed. The appropriate time point frequency depends on the patient's condition, the therapy factors controlled, and the correlation factors measured. Suitable time point frequencies include, for example, approximately every second, approximately every 10 seconds, approximately every minute, approximately every 15 minutes, approximately every 30 minutes, approximately every 2-60 minutes, approximately every hour, approximately every other hour, approximately every 3 hours, approximately every 4 hours, approximately every 6 hours, approximately every 12 hours, approximately every 2-24 hours, approximately every day, approximately every other day, approximately every week, approximately every other week, and approximately every monthly.

As used herein, “correlation” is not used in a formal statistical sense, where one is dealing with two variables. In statistical terms, an association between data sets is “experimental” (one variable) or “correlational” (two variables). Here, “correlation” simply means a relationship between two or more sets of data. The correlation can be positive or negative. A negative correlation does not necessarily mean an undesirable relationship.

For ease of interpretation, it is preferred to have positive numbers indicate desired results.

By “the corresponding difference” it is meant the correlation factor difference from a suitable, predetermined pair of time points. Generally, the same two time points that were used to calculate the difference in therapy elements, will be used to calculate a difference in correlation factors. However, another suitable pair of correlation factor time points, offset by a fixed time interval from the therapy element pair can also be used in accordance with the invention. The use of such an alternative source of the pair of correlation factor time points is referred to herein as “time offset analysis” and takes into account any potential delayed effects of therapies.

By “time intervals” it is meant a subset of the entire time course made up of two or more successive pairs of time points.

By “rolling average” it is meant the successive averages for a series of intervals of constant size. Accordingly, a rolling average drops the oldest data for each item of new data.

By “processed difference” it is meant the result of the application of a function that transforms the calculated differences in therapy elements and correlation factors to digitized or normalized forms (i.e., digitizing or normalizing the differences.)

By “digitizing” it is meant a transformation of a calculated difference in therapy elements and correlation factors, which substitutes either +1 for positive change (e.g., dayn+1 greater than dayn), 0 (zero) for no change, and −1 for negative change (e.g., dayn+1 less than dayn). Sign (+ or −) is relative and is intended so here. For example, no difference in substance is intended between calculating (x−y) or (y−x), even though the resulting differences may have opposite signs. One of ordinary skill in the art is presumed competent to track the significance of sign. Generally, later data is greater than earlier data if a patient is improving and subtracting earlier data from later data produces a positive difference. If this were not the case, e.g. for fever, one could reverse the order of subtraction to produce a positive difference or simply remember that negative numbers represent desired change when monitoring fever.

By “normalizing” it is meant processing the data so that different therapy elements and correlation factors can be compared, regardless of the actual values measured for the individual therapy elements and correlation factors. In particular, a preferred normalization technique is the generation of a decimal fraction representing the absolute value of the change (difference) in a given metric between two time points, preferably two successive time points:

|(Aⱼ−Aⱼ₋₁)|/Aⱼ₋₁.

Alternatively, normalization can yield a positive or negative percent change in a metric between two time points, preferably two successive time points:

100×[(Aj−Aⱼ₋₁)/Aⱼ₋₁].

Normalization may also comprise the calculation of the reciprocal of any measured change, for example, taking the reciprocal of the decimal fraction of the absolute value of change in a therapy element from one time point to the next. To avoid division by zero, any Aⱼ₋₁ value of zero can, optionally, be set to 0.01.

By “modified in accordance with the display” it is meant that a therapy element or a correlation factor will be changed so as to potentiate the therapy, i.e., optimize the overall therapy so as to best produce the most desirable patient outcome. By “the most desirable patient outcome” it is meant best possible quality of life, preferably including lowest possible level of morbidity and therapy-related side effects, more preferably including the longest possible survival, and most preferably including cure at the lowest possible treatment cost.

By “modified in accordance with the displayed rolling averages and Systems Functions” it is meant that a therapy element or a correlation factor will be changed based on the trends observable in the rolling averages and using the predictions resulting from the application of the Systems Functions so as to potentiate the therapy, i.e., optimize the overall therapy so as to best produce the most desirable patient outcome. For example, the therapy elements associated with the most positive rolling averages are modified based on predictions generated by the Systems Functions derived from those rolling averages.

By “System Function” it is meant a factor by which a therapy element can be multiplied to yield a correlation factor. Such a system function can be derived by linear regression analysis of a therapy element-correlation factor curve.

By a “narrow therapeutic band” it is meant the narrowest range, from maximum to minimal therapy dose, level or intensity that produces cure without therapy-related mortality or morbidity. Typically, two or more potentiating elements form the core of a therapeutic protocol they create a narrow therapeutic band. The “origination point” of the narrow band is the point of minimum dosing of any element below which therapeutic effect decreases to the extent that it no longer controls disease. The “end point” of the narrow band is the point of maximum dosing of any element, above which there is diminishing returns or no change in therapeutic value, but a nonlinear change in adverse side effects.

The narrow band for an individualized therapeutic protocol can expand and contract, as well as shift Up and down, depending on certain factors, such as:

the amount of time since diagnosis of the disease,

the amount of time since initiation of therapy,

the state of disease progression, including relapse and refractory conditions,

general health,

remission time, and

addition or subtraction of elements, or changes in other health parameters, mind-body initiatives and other environmental factors.

The origination point of the narrow band can be determined through observation of visual improvement in disease effects and metrics based upon medical tests that show progression, stability or remission of disease parameters. The end point of the narrow band is determined through observation of visual side effects and metrics based upon medical tests that would show non-observable side effects, such as reduction in immune function or changes in metabolite balance.

### II. POTENTIOLOGY™

The study and application of potentiation, as carried out herein by the assignee of this application under the name “POTENTIOLOGY™,” whereby multiple elements within an environment amplify or synergize each other to create a disproportionate or geometric result. These elements interrelate through a system of feedback loops to produce a potentiating dynamic that results in achieving success at a greatly accelerated rate. Applying the POTENTIOLOGY™ driven treatment protocol to serious diseases results in the production of a therapeutic protocol that can be characterized by the following:

1. Minimum dosing to therapeutic effect, which is patient-specific, and sustainable, as it engenders patient stability. This is in contrast to maximum dosing to patient intolerance, which is typically generalized, providing uniform dose levels to all patients, and that is often unsustainable due to side effects that destabilize the patient.

2. Protocol simplicity, focusing on the fewest number of therapy elements that make up the treatment protocol, as possible

3. Time, as a constitutive component of the protocol, to allow all of the therapy elements to reach their maximum effect at minimum dose. This is as opposed to viewing time as the “enemy,” considering it as a constraint that requires the heavy dosing of limited therapy elements

4. Achieving a geometric or disproportionate therapeutic effect through the potentiation or synergizing of key therapy elements. This approach makes minimum dosing both possible and effective, with the result, conceptually, of creating a symmetrical balance with the operating mechanisms of the disease. This symmetry is required for protocol effectiveness and sustainability.

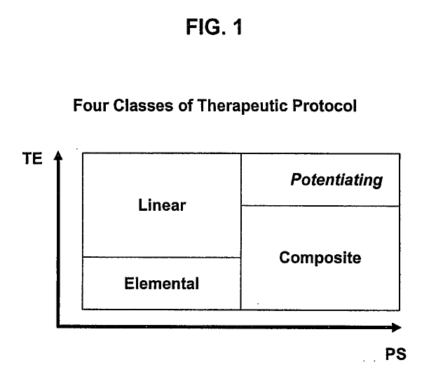

Generally, treatment protocols can be categorized into four classes (FIG. 1 depicts the four classes of protocols, within the two dimensions of Therapeutic Effect (TE) and Patient Stability (PS)):

Elemental Protocol—principally, a single therapy element, often given in maximum tolerated doses to insure the greatest effect in a short period of time. In the case of cancer, this approach is often characterized by a results pattern of disease reduction, remission and relapse, with secondary side effects that require additional therapies that seek to stabilize the patient.

Linear Protocol—a number of therapy elements that are applied sequentially, over time, often as part of a trial-and-error effort. As with the Elemental Protocol, the result is often a pattern of disease reduction, remission and relapse, with secondary side effects that require additional therapies.

Composite Protocol—the combination of diverse, yet often complementary, therapy elements that remain somewhat fixed during the course of treatment. Forming a common denominator with the Elemental and Linear Protocols, the pattern of disease reduction, remission and relapse is often apparent, but to a lesser extent, as treatment failure rates are lower and disease progression is slower with fewer side effects due to less than maximum dosing.

Potentiation Protocol—the combination of synergizing therapy elements that interrelate and are interdependent on each other to form an adaptive dynamic system that is conceptually symmetrical to the synergizing mechanisms of cancer that form an adaptive cancer dynamic system.

The components of the process used to develop a POTENTIOLOGY™ driven treatment protocol are:

1. Primary therapy elements—drugs, supplements, diet, exercise and other agents/factors, including their method of administration, that potentiate each other or that have the potential to potentiate each other to create a geometric or disproportionate therapeutic effect, as the core of a Potentiation Protocol.

2. Secondary therapy elements—drugs, supplements, diet, exercise and other agents/factors, including their method of administration, that directly or indirectly support the improvement or potential improvement of the patient, thus supporting the core or primary therapy elements of a Potentiation Protocol.

3. An ongoing cause and effect mathematical analysis taken to establish the existence of any connectivity between all aspects of a treatment protocol, including therapy elements and correlation factors. The cause and effect analysis will also assist in determining the minimum dosing levels necessary to create therapeutic effect, based upon the identification of cause and effect relationships between specific therapy elements and other therapy elements, therapy elements and correlation factors, and specific correlation factors and other correlation factors.

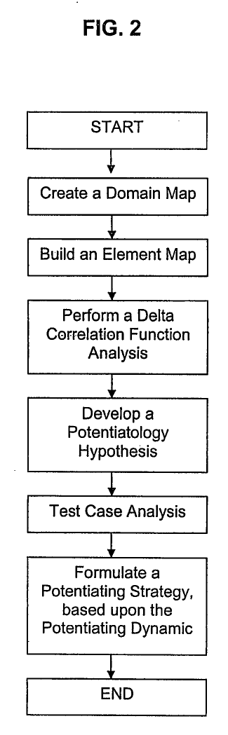

The development of a POTENTIOLOGY™ driven treatment protocol is an ongoing process which can be used to continuously optimize therapy or can be stopped after achieving outcome goals. In general, the development of a POTENTIOLOGY™ driven treatment protocol comprises (see flow chart, FIG. 2). First, researching primary therapy elements, secondary therapy elements, and correlation factors. This results in a characterization of the environment, in terms of a grouping of primary therapy elements, secondary therapy elements, and correlation factors in putative operational domains and the processes that function within and between those domains. The result is referred to as a “Domain Map.” Then, specific therapy elements and correlation factors are selected for use in the timecourse producing an “Element Map” (FIG. 2).

The chosen therapy elements and correlation factors are monitored over time (FIG. 2) and data is collected relative to the individual therapy elements (Aj) and the correlation factors (Bj) at time points (j) in the timecourse, allowing for the compilation of pairs of data, (Aj,Bj).

“Delta Correlation Function Analysis” (FIG. 2) is performed using a plurality of pairs by computing change in therapy element (Aj−Aj−1) and change in correlation factor (Bj−Bj−1) producing pairs of differences. Each corresponding pair of differences is multiplied (preferably after normalization and/or digitization) to obtain a correlation product (Aj−Aj−1)×(Bj−Bj−1). A rolling average of the correlation products is displayed as a table, graph or chart, from which one can see positive, negative, or no correlation. The rolling average includes several periods of administration. Products can be calculated from more than two pairs of data, if necessary, to provide a phase correction (time offset) in the correlation product.

A hypothesis is developed, based upon possible feedback loops, as to which therapy elements might potentiate each other to produce a multiplicative effect. This is referred to as a “POTENTIOLOGY™ Hypothesis” (FIG. 2). A series of test modifications are undertaken to prove the POTENTIOLOGY™ Hypothesis and to systematically identifying feedback loops. Ultimately, verified POTENTIOLOGY™ Hypotheses produce a specific strategy, the Potentiating Strategy.

### III. Applying POTENTIOLOGY™ to Medical Treatment

The invention provides methods and computer program products comprising a computer-readable media having thereon computer-executable instructions for the performance of a method for potentiating a therapy in a patient, wherein the method for potentiating a therapy in a patient comprising the steps of:

(a) selecting a plurality of therapy elements;

(b) selecting a plurality of correlation factors;

(c) collecting data relative to the therapy elements and the correlation factors at a plurality of time points in a timecourse; and

(d) analyzing the data by

(i) calculating a difference between the therapy elements for each successive pair of time points and a difference between the correlation factors for each successive pair of time points;

(ii) processing the differences by digitizing or normalizing each of the calculated differences, with or without also converting the resulting therapy elements into their reciprocals;

(iii) calculating a product of the processed (digitized or normalized) difference for each therapy element from each successive pair of time points multiplied by the corresponding digitizing or normalizing difference for each correlation factor from each successive pair of time points;

(iv) calculating respective rolling averages of the products over the timecourse;

(v) displaying one or more of the rolling averages; and

(vi) modifying the therapy in accordance with the display.

Generally, the time point pairs used for the calculation of the differences will be the same for the therapy elements and correlation factors, i.e, the “corresponding” differences will be from the same time after the beginning of the study. However, time offset correlation factors can also be used in accordance with the invention. Thus, while methods in accordance with the invention will typically multiply the difference between the therapy elements at time points t and t+1 by the difference between the correlation factors at a time points t and t+1; the invention also provides for the multiplication of difference between the therapy elements a time points t and t+1 by the difference between the correlation factors at a time points t+n and t+n+1, where “n” is a fixed, predetermined integral value (i.e., is offset in time).

In some embodiments, the invention provides computer program products comprising computer-readable media having thereon computer-executable instructions for the performance of a method wherein calculating the normalized therapy element values as decimal fractions or percent changes, and optionally, further converting the resulting therapy element decimal fractions to their reciprocals.

The invention provides computer program products comprising computer-readable media having thereon computer-executable instructions which provide a recommendation for a modification of the therapy in accordance with the display based on the data analysis performed. Such recommendations can, for example, be based on the program's detection of the greatest therapy element-correlation factor products, the results of data reduction, closeness to desired outcome metrics, formulas or predetermined modification rules. Such computer program generated recommendations can be directed to the modification of one or more therapy elements, preferably 2 or more therapy elements, more preferably 3 or more therapy elements, even more preferably 4 or more therapy elements, even more preferably 5 or more therapy elements, and most preferably 6 or more therapy elements and/or one or more correlation factors, preferably 2 or more correlation factors, more preferably 3 or more correlation factors, even more preferably 4 or more correlation factors, even more preferably 5 or more correlation factors, and most preferably 6 or more correlation factors.

The use of displays which highlight therapy elements whose alteration produces disproportion results allows the invention to present a complex data set in a rapidly understandable form. Any suitable tabular or graphic presentation can be used to display the rolling averages. Graphical or tabular displays in accordance with the invention can depict or present the rolling averages and/or the data on which the rolling averages are based, either before or after data reduction. Further, graphical or tabular displays in accordance with the invention can depict or present one, many (e.g., at least about 3, at least about 10, at least about 20, at least about 30, at least about 50, at least about 100, at least about 200, at least about 500 or at least about 1,000) or all of the therapy element/correlation factor pairs in a single view. In preferred embodiments the rolling averages, and any other data, are displayed both in graphical and tubular forms. In the most preferred embodiments the rolling averages, and any other data, are displayed both in graphical and tubular forms wherein the presentations provide the linkage of the presentations such that the user can “click” on a link to move from a portion of one presentation of the data to the corresponding portion of another presentation of the data.

Even with effective display, the shear volume of data resulting from the potentiation process can be overwhelming. Accordingly, in preferred embodiments of the invention, a data reduction step is inserted between step (v) calculating a rolling average of the products over all of the timecourse, and step (vi) displaying the rolling averages, and wherein the reduced rolling averages are displayed. In other words, the number of rolling averages for display is reduced by the data reduction step so that a less complex data set is presented. By “data reduction step” it is meant a protocol, process, program or algorithm which reduces the complexity of the data while emphasizing the most variable data and minimizing noise. Any suitable data reduction protocol, process, program or algorithm can be used in accordance with the invention. Preferred data reduction protocols, processes, programs or algorithms include, for example, cluster analysis, principle component analysis (“PCA”), factor analysis, discriminant function analysis, multidimensional scaling, and combinations thereof. Such suitable data reduction protocols, processes, programs or algorithms are well known to those of ordinary skill in the art and can be used with either the methods or computer program products provided by the invention.

For example, cluster analysis classifies a set of observations into two or more mutually exclusive unknown groups based on combinations of interval variables. Data clustering algorithms can be hierarchical or partitional. Hierarchical algorithms find successive clusters using previously established clusters, whereas partitional algorithms determine all clusters at once. Hierarchical algorithms can be used in accordance with the invention to, for example, identify closely related therapy elements so that potentially redundant modification is not undertaken.

Partitional algorithms, are preferred for use in accordance with the invention and include, e,g, K-means clustering, self-organizing maps, graph-theoretic methods and the like. Partitional algorithms can be used in accordance with the invention to, for example, identify groups or “domains” of related therapy elements so that only one therapy element form each group is modified. Suitable cluster analysis computer programs are available from commercial sources such as StatSoft, Inc., Tulsa, Okla. 74104; Advanced Clustering Technologies, Inc., Kansas City, Kans. 66103; and American Heuristics Corporation, Triadelphia, W. Va. 26059.

PCA is particularly preferred data reduction technique and is designed to capture the variance in a dataset in terms of principle components. PCA reduces the dimensionality of the data to emphasize the most important (i.e. defining) parts while simultaneously filtering out noise. “Principle Components” are a set of variables that define a projection that encapsulates the maximum amount of variation in a dataset and is orthogonal (and therefore uncorrelated) to the previous principle component of the same dataset. Further, PCA can identify the individual variables whose behavior contributes the most to each Principle Component. Accordingly, PCA can be used in accordance with the invention to both identify groups of independently varying correlation factors and the individual therapy elements that produce the greatest effect. For example, the therapy elements that contribute the most to each Principle Component can be recommended for modification by computer programs used in accordance with the invention. Suitable PCA software applications are available from commercial sources such as StatSoft, Inc., Tulsa, Okla. 74104; Addinsoft, USA, New York, N.Y. 10013; statistiXL Broadway—Nedlands, Western Australia, 6009; and as shareware

Computer programs which can execute other suitable data reduction techniques, such as actor analysis (or common factor analysis), discriminant function analysis, and multidimensional scaling are also commercially available from vendors such as IBM Corporation, Armonk, N.Y. 10504-1722; SPSS Inc., Chicago, Ill. 60606; Partek Incorporated, St. Louis, Mo. 63141; SAS Institute Inc., Cary, N.C. 27513-2414; Rosella Software, Sydney, Australia; Alyuda Research, Los Altos, Calif. 94024; Peltarion HB, Stockholm, Sweden; and Greenplum, San Mateo, Calif. 94403.

Suitable data reduction protocols, processes, programs or algorithms for use in accordance with the invention can further identify and display the therapy elements producing the greatest variation in the data. This displaying step can be followed by a step of modifying one or more therapy elements producing the greatest variation shown in the data in accordance with the display.

Methods in accordance with the invention include, for example, wherein the timecourse has at least three time points, preferably at least five time points, and more preferably least five time points and wherein the respective rolling averages includes data from at least two intervals, preferably at least three intervals, and more preferably at least four intervals. Nevertheless, methods done in accordance with the invention can have timecourses with more than five time points, more than 15, more than 25, more than 50, more than 100, more than 500 or more than 1,000 time points. Methods done in accordance with the invention can also have respective rolling averages that include data from more than four, more than 10, more than 20, more than 25, more than 50, more than 100, more than 500 or more than 1,000 intervals.

To additionally facilitate the potentiation of therapies directed one or more patients with a particular condition, the invention provides embodiments wherein the methods and computer program products are employed further comprising a step (vii) wherein one or more therapy elements are modified in accordance with the display and/or one or more correlation factors are modified in accordance with the display.

Further, the invention provides embodiments wherein the methods and computer program products in accordance with the invention include a step wherein one or more, preferably 2 or more, more preferably 3 or more, even more preferably 4 or more, even more preferably 5 or more, and most preferably 6 or more therapy elements are modified in accordance with the display and/or one or more, preferably 2 or more, more preferably 3 or more, even more preferably 4 or more, even more preferably 5 or more, and most preferably 6 or more correlation factors are modified in accordance with the display of unreduced or reduced data.

In preferred embodiments of the invention the method is repeated, either independently of or using computer program products in accordance with the invention, one or more times modifying one or more therapy elements on each repetition of the method. Such repetitions done in accordance with the invention include embodiments wherein the method is repeated until at least one predetermined outcome metric is satisfied, preferably at least 2 predetermined outcome metrics are satisfied, more preferably at least 3 predetermined outcome metrics are satisfied, even more preferably at least 4 predetermined outcome metrics are satisfied, even more preferably at least 5 predetermined outcome metrics are satisfied, and most preferably at least 10 predetermined outcome metrics are satisfied.

The invention provides for the modifications to be directed by a skilled clinician employing reasonable judgment. Alternatively, invention provides for modifications based on predetermined “modification rules.” For example, the kind and/or degree of modification for each therapy element or correlation factor can be limited. Additionally, for example, the modification rules can use formulas for the modification of therapy elements based on values for correlation factors or the change in values for correlation factors. Such formulas can by computer programs in accordance with the invention to generate recommendations for modifications of therapy factors.

The invention provides methods and computer program products whereby the method is repeated until at least one predetermined outcome metric is satisfied. By “satisfied” it is meant that based on reasonable clinical judgment and objective criteria an acceptable value for the metric has been achieved. Alternatively, by “satisfied” it is meant that the metric has achieved a value of within about 50%, preferably within about 40%, more preferably within about 33%, even more preferably within about 25%, even more preferably within about 20%, even more preferably within about 15%, even more preferably within about 10%, and most preferably within about 5% of a predetermined goal value. It is within the skill of the ordinarily skilled clinician to determine suitable goal values. In addition, the invention provides computer program products which can determine the degree to which predetermined outcome metrics have been approached, including whether one or more predetermined outcome metrics have been satisfied, and display the results of this determination.

Suitable outcome metrics include, for example, measuring a physiologic parameter, measuring patient performance status, measuring patient symptom, measuring patient reported subjective factor, measuring family reported subjective factor, measuring side effects, measuring complications, measuring cost or measuring compliance with a narrow therapeutic band. Physiologic parameters which can be used as outcome metrics in accordance with the invention include, for example, weight, temperature, heart rate, blood pressure, respiratory rate, physical examination results (e.g., from inspection, palpation, percussion and auscultation, neurological or orthopedic manipulations), blood chemistry results, blood counts, blood gas levels, x-ray results, MRI results, sonography results, biopsy results or any other suitable clinical measurement. In general, suitable correlation factors are suitable outcome metrics. Accordingly, a subset of the correlation factors may be used outcome metrics. However, the invention envisions that there will be significantly more correlation factors than outcome metrics.

The normalized Correlation Function Value can be based upon the calculated products of the processed difference for each therapy element for each successive pair of time points multiplied by the processed difference for successive pairs of correlation factor time points, wherein each correlation factor pair of time points is offset in time by a fixed interval from each therapy element pair. Thus, changes in therapy elements and their corresponding impact on correlation factors, as a function of time to account for different times to therapy element therapeutic effect and metabolism rates, are established. The recognition of an offset pattern then allows for the use of linear regression analysis of the data collected during a timecourse to generate hypothetical Systems Functions, which incorporate the dose and dosing cycle. POTENTIOLOGY™ between therapy elements is established when therapeutic effect exists at dose levels below that of published or recommended individual therapeutic element dose levels or when therapy elements are administered according to a dosing cycle, such as 2 days on, 1 day off, to effectively fall below published or recommended dose levels. The Systems Functions can then be used to design hypotheses used modify therapy.

The invention provides methods and computer program products for the potentiation of the therapy of any suitable disease or condition including, for example, neoplastic diseases, infectious diseases, autoimmune diseases, metabolic diseases, psychiatric diseases, and combinations thereof, in a human or non-human patient.

The invention thus provides methods and computer program products for use in the therapy of any suitable neoplastic disease including, for example, benign tumors and hyperplasias, premalignant conditions, and malignancies such as carcinomas, sarcomas, teratomas, lymphomas, leukemias, and plasma cell dyscarsias. More specifically, suitable neoplasias include, for example, oral cavity tumors, pharyngeal tumors, digestive system tumors, the respiratory system tumors, bone tumors, cartilaginous tumors, bone metastases, sarcomas, skin tumors, melanoma, breast tumors, the genital system tumors, urinary tract tumors, orbital tumors, brain and central nervous system tumors, gliomas, endocrine system tumors, thyroid tumors, esophageal tumors, gastric tumors, small intestinal tumors, colonic tumors, rectal tumors, anal tumors, liver tumors, gall bladder tumors, pancreatic tumors, laryngeal tumors, tumors of the lung, bronchial tumors, non-small cell lung carcinoma, small cell lung carcinoma, uterine cervical tumors, uterine corpus tumors, ovarian tumors, vulvar tumors, vaginal tumors, prostate tumors, prostatic carcinomas, testicular tumors, tumors of the penis, urinary bladder tumors, tumors of the kidney, tumors of the renal pelvis, tumors of the ureter, head and neck tumors, parathyroid tumors, Hodgkin's disease, Non-Hodgkin's lymphoma, multiple myeloma, leukemia, acute lymphocytic leukemia, chronic lymphocytic leukemia, acute myeloid leukemia, chronic myeloid leukemia, benign prostatic hyperplasia, polyps, papillomas, hyperplasias including endometrial hyperplasia, myelodysplasic syndromes, atypical hyperplasias, and carcinomas in situ.

Accordingly, appropriate therapy elements include, for example, dosages or drug levels of chemotherapeutic agents (e.g., docetaxel, paclitaxel, taxanes and platinum compounds), antifolates, antimetabolites, antimitotics, DNA damaging agents, proapoptotics, differentiation inducing agents, antiangiogenic agents, antibiotics, hormones, peptides, antibodies, tyrosine kinase inhibitors, biologically active agents, biological molecules, adriamycin, ansamycin antibiotics, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, meplhalan, methotrexate, rapamycin (sirolimus), mitomycin, mitotane, mitoxantrone, nitrosurea, paclitaxel, pamidronate, pentostatin, plicamycin, procarbazine, rituximab, streptozocin, teniposide, thioguanine, thiotepa, taxanes, vinblastine, vincristine, vinorelbine, taxol, combretastatins, discodermolides, transplatinum, anti-vascular endothelial growth factor compounds (“anti-VEGFs”), anti-epidermal growth factor receptor compounds (“anti-EGFRs”), antiangiogenic agents, antineoplastic biologics, and radionuclides

The invention provides methods and computer program products for use in the therapy of any suitable infectious disease including viral, bacterial, fungal, and polymicrobial infections, infestations, and colonizations. More specifically, suitable infectious agents include, for example, Pseudomonas aeruginosa, Escherichia coli, Enterococcus hirae, Acinetobacter baumannii, Acinetobacter species, Bacteroides fragilis, Enterobacter aerogenes, Enterococcus faecalis, Vancomycin resistant-Enterococcus faecium (VRE, MDR), Haemophilus influenzae, Klebsiella oxytoca, Klebsiella pneumoniae, Micrococcus luteus, Proteus mirabilis, Serratia marcescens, Staphylococcus aureus, Staphylococcus epidermidis, Staphylococcus haemolyticus, Staphylococcus hominis, Staphylococcus saprophyticus, Streptococcus pneumoniae, Streptococcus pyogenes, Salmonella choleraesuis, Shigella dysenteriae, and other susceptible bacteria, as well as yeasts, e.g., Trichophyton mentagrophytes, Candida albicans and Candida tropicalis. The methods and computer program products taught herein can be used in accordance with the invention to treat viruses including, e.g., adenovirus, human immunodeficiency virus (HIV), rhinovirus, influenza (e.g., influenza A), hepatitis (e.g., hepatitis A), coronavirus (responsible for, e.g., Severe Acute Respiratory Syndrome (SARS)), rotavirus, avian flu virus, respiratory syncytial virus, herpes simplex virus, varicella zoster virus, rubella virus, and other susceptible viruses.

Accordingly, appropriate therapy elements include for example, dosages or drug levels of aminoglycosides (amikacin, gentamicin, kanamycin, netilmicin, tobramycin, treptomycin, azithromycin, clarithromycin, erythromycin, erythromycin estolate/ethyl-succinate/gluceptate/lactobionate/stearate), beta-lactams such as penicillins (e.g., penicillin G, penicillin V, methicillin, nafcillin, oxacillin, cloxacillin, dicloxacillin, ampicillin, amoxicillin, ticarcillin, carbenicillin, mezlocillin, azlocillin and piperacillin), or cephalosporins (e.g., cephalothin, cefazolin, cefaclor, cefamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, and cefsulodin), carbapenems (e.g., imipenem), monobactams (e.g., aztreonam), quinolones (e.g., fleroxacin, nalidixic acid, norfloxacin, ciprofloxacin, ofloxacin, enoxacin, lomefloxacin and cinoxacin), tetracyclines (e.g., doxycycline, minocycline, tetracycline), glycopeptides (e.g., vancomycin, teicoplanin), chloramphenicol, clindamycin, trimethoprim, sulfamethoxazole, nitrofurantoin, rifampin, mupirocin, cationic peptides, posaconazole, voriconazole, ketoconazole, fluconazole, itraconazole, saperconazole, neticonazole, oxiconazole, isoconazole, sulconazole, terconazole, ravuconazole, capsofungin, tioconazole, acyclovir, ganciclovir, fancyclovir, valacyclovir, and antiretrovirals, including HIV protease inhibitors, nucleoside and non-nucleoside HIV reverse transcriptase inhibitors, and HIV fusion inhibitors.

Especially suitable infectious diseases for the use of the invention include, for example, bacteremia, fungemia, abscess formation, gastric H. pylori, malaria, viral encephalitis virus, and diseases caused by HTLV-I, HIV, HBV, HCV or a mycobateria.

The invention provides methods and computer program products for use in the therapy of any suitable autoimmune disease including, for example, lupus, arthritis, mixed connective tissue disease, inflammatory bowel disease, graph versus host disease and transplant rejection; any suitable metabolic disease or condition including, for example, diabetes, metabolic syndrome and hyperlipidemia; and any suitable psychiatric disease including for example a psychosis, an anxiety disorder, an affective disorder, an attention disorder, and a personality disorder. Accordingly, appropriate therapy elements include, for example, dosages or drug levels of antianxiety agents, oral hypoglycemics, insulins, steroid hormones, anti-inflammatories, antipsychotic agents, cognitive enhancers, cholesterol-reducing agents, anti-atherosclerotic agents, antiobesity agents, autoimmune disorder agents, anti-Parkinsonism agents, anti-Alzheimer's disease agents, anti-depressants, glycogen phosphorylase inhibitors, and cholesterol ester transfer protein inhibitors

Particularly preferred embodiments of the invention provide methods and computer program products for the potentiation of the therapy of acute conditions. The therapy of any suitable acute condition can be potentiated in accordance with the methods and computer program products provided by the invention including, for example, birth, sepsis, stroke, myocardial ischemia, neurogenic shock, shock trauma, surgery, anesthesia, and combinations thereof. Accordingly, appropriate therapy elements include for example, dosages or levels of coagulation factors, fluids, blood products, tocolytics, uterine stimulants, steroids, oxygen, analgesics, anesthetics, antihypertensives, antianxiety agents, anticlotting agents, anticonvulsants, insulins, antihistamines, antitussives, calcium channel blockers, beta blockers, anti-inflammatories, antipsychotic agents, nitrates, cardiac inotropic agents, anti-apoptics, diuretics, antibiotics, anti-depressants, antiviral agents, and antifungal agents.

Those of ordinary skill will recognize that, over time, there can be changes in the selection of therapy elements, correlation factors, dose, and administration are made based upon the analysis of correlation products. New data is collected and analyzed as the process repeats itself to produce a therapy designed to move the patient's condition to chronic and manageable, where, e.g., in the case of cancer, apoptosis of cancer cells may occur.

Those of ordinary skill will also recognize that the methods and computer program products taught herein can be use in accordance with the invention for the potentiation of the therapy of groups of patients. When groups of patients are treated, therapy elements and correlation factors are collect and analyzed both within individuals and across different individuals. Suitable patient group sizes include at least about 3, at least about 5, at least about 10, at least about 20, at least about 50, at least about 100, and at least about 200 patients.

The invention thus provides a method for analyzing data to provide minimum dose for therapeutic effect and to correlate treatment and symptoms. The invention also provides a method for analyzing treatment that can accommodate changes in treatment with changes in the condition of the patient and allows time for the treatment to reach maximum effect at minimum dose, unlike elemental therapies. The result is a therapy that combines elements to obtain a disproportionate, positive effect compared with elemental, linear, or composite therapies.

Having thus described the invention, it will be apparent to those of skill in the art that various modifications can be made within the scope of the invention. For example, while described for the sake of simplicity as a product of two numbers, the correlation factor can be based more than one pair of numbers. Whether or not to digitized, selecting an averaging period, and determining phase adjustments are readily and rapidly determined empirically simply by viewing the charts produced by a spreadsheet program. While specific examples of primary elements, secondary elements, and correlation factors are given, they relate to the particular embodiment described. Both data in a pair need not be either digitized or undigitized. That is, one datum can be digitized and the other datum not digitized. The administration period varies with the treatment and is a consideration when averaging or shifting phase.

The following examples further illustrate the invention but, of course, should not be construed as in any way limiting its scope.

### EXAMPLE 1

This example demonstrates the application of POTENTIOLOGY™ to a theoretical multi-element, complex environment which mimics drug therapy in a patient.

1. Building an Element/Factor Map

The first tasks are to determine which systems contribute to an environment, analyze what is known about the systems, and construct lists or “maps” of the systems' components showing how they are thought to relate to one another. “Elements” are the inputs and “factors” are outputs of systems. An output is a result achieved when an input element is multiplied by a particular “System Function.” When multiple inputs and outputs are recorded, an output to input curve or System Function curve can be constructed and the System Function determined by linear regression analysis. For example, the invention provides for determining the optimum dose of a drug as part of a treatment protocol. The dose of the drug would represent the input or “therapy element” and the side effects and/or therapeutic effects representing outputs or “correlation factors.”

Within a patient, there are often multiple systems, or “domains,” such as organs that combine to produce the patient's overall condition. POTENTIOLOGY™ is applied to a patient's care on both an intra and inter domain basis. Initially, it will may not be clear as to which variables are input or outputs or resultants, based upon cause and effect relationships. Therefore, in constructing an Element/Factor Map it is important to identify all possible elements or variables and the make of the domains or the domains themselves may change as the system is studied.

2. Performing a Delta Correlation Function Analysis

“Delta Correlation Function Analysis” is the process which determines how the change in a therapy element (the therapy element “1a”) is correlated with the change in a correlation factor (the correlation factor “2a”). Delta Correlation Function Analysis begins with the construction of a matrix for each domain by:
a. Identifying all appropriate therapy elements and correlation factors within a specific domain, for example “1a, 2a and 3a.”
b. Recognizing that each therapy element or correlation factor is a variable—record the value of each such variable for all available samplings. For example, record the values of therapy element 1a, 2a and 3a for days 1, 2 and 3, represented as 1a-1, 1a-2, 1a-3, 2a-1, 2a-2, 2a-3, 3a-1, 3a-2 and 3a-3 as shown in the following table:

| Element | Day 1 | Day 2 | Day 3 |
|---|---|---|---|
| 1a | 1a-1 | 1a-2 | 1a-3 |
| 2a | 2a-1 | 2a-2 | 2a-3 |
| 3a | 3a-1 | 3a-2 | 3a-3 |

Similarly, the following is an exemplary table of data for element 1a, adding factor 2a over the 3 points in time; t1, t2 and t3:

| Element 1a | | | Factor 2a | | |
|---|---|---|---|---|---|
| Day 1 | Day 2 | Day 3 | Day 1 | Day 2 | Day 3 |
| 1a-1 | 1a-2 | 1a-3 | 2a-1 | 2a-2 | 2a-3 |

Second, a Delta Correlation Function Analysis is done to determine which correlation factors are sensitive to which therapy elements. The differences between the values of each therapy element and each correlation factor from one time point to the next is calculated. These differences are digitized (i.e, increase in a variable is represented by +1 and a decrease in a variable in represented by −1).

A typical analysis would include:

1. Determining Therapy Element Differences for Day 2, Relative to Day 1:

Is 1a-2 greater than, equal to or less than 1a-1?

Is 1a-3 greater than, equal to or less than 1a-2?

If 1a-2>1a-1, the Day 2 Element 1a=+1

If 1a-2=1a-1, the Day 2 Element 1a=0

If 1a-2<1a-1, the Day 2 Element 1a=−1

The same is true of Day 3, relative to Day 2

2. Determining Correlation Factor Differences for Day 2, Relative to Day 1:

If 2a-2>2a-1, the Day 2 Factor 2a=+1

If 2a-2=2a-1, the Day 2 Factort 2a=0

If 2a-2<2a-1, the Day 2 Factort 2a=−1

The same is true of Day 3, relative to Day 2

Next, the “Correlation Function Values” are determined. The Correlation Function Value for a specific digitized element/factor pair for a particular day is determined by multiplying the therapy element's digitized difference for that pair of days times the correlation factor's digitized difference for same pair of days. Thus, when multiplying the Day 1-2 element 1a difference (+1, 0, −1) by the Day 1-2 factor 2a difference (+1, 0, −1), the Correlation Function Value (CFVa2) will equal +1, 0 or −1.

This approach is taken for a specific element relative to a specific factor for every time point pair, thus reflecting the total number of samples:

time point pair 1 (1a-2)×(2a-2)=CFVa2

time point pair 2 (1a-3)×(2a-3)=CFVa3

•

•

•

time point pair n (1an)×(2an)=CFVan

The Correlation Function Values are then displayed in a table or graphically

3. Determine the Rolling Averages

Finally, while the Correlation Function Values tends to factor out “noise” or spurious fluctuations in data, the Correlation Function Values should be “normalized” to best establish connectivity between elements and factors. This can be achieved for a particular element/factor pairs, such as element 1a and factor 2a, by adding the Correlation Function Values for all samples and dividing the total by the number of samples:

Normalized Correlation Function (element 1a,factor 2a)=(CFVa2+CFVa3+CFVan)/n.

The result is an indication of relationship or “connectivity” as follows:

| Positive Connectivity | No Connectivity | Negative Connectivity |
|---|---|---|
| If, the Normalized | If, the Normalized | If, the Normalized |
| Correlation Function | Correlation Function | Correlation Function |
| Value is greater that | Value is close to zero, it | Value is less than zero, |
| zero, it suggests a | suggests no correlation | it suggests a negative |
| positive correlation | between therapy | correlation between |
| between therapy | element 1a and | therapy element 1a and |
| element 1a and | correlation factor 2a | correlation factor 2a |
| correlation factor 2a | | |

Cognizant that connectivity may be altered during the course of therapy, a rolling average of the normalized Correlation Function Value is graphically displayed to facilitate the recognition of patterns of connectivity. Therapy can then be altered in accordance with the thus determined connectivity between therapy elements (input) and correlation factors (output). Moreover, hypotheses as to the operative System Function producing the connectivity between therapy elements (input) and correlation factors (output) can be formulated and tested against the collected

### EXAMPLE 2

This example demonstrates the applicability of the invention to the treatment of an animal.

The invention is exemplified in the context of multiple myeloma in a cat, in which the disease is particularly aggressive. At the time of diagnosis the cat had a projected survival time of less than thirty days. However, the cat in this example was alive and stable as a result of employing the invention's methods over two years after initial diagnosis.

Multiple myeloma is a progressive malignant blood disease. It is a cancer of the plasma cell, an important part of the immune system that produces antibodies. Multiple myeloma is characterized by proliferation of plasma cells, where an excessive number of these plasma cells invade bone marrow and other organs, increase vascularization around the tumor, and by the overproduction of intact monoclonal (M) immunoglobulin or dysfunctional antibodies. Hypercalcemia, anemia, renal damage, increased susceptibility to bacterial infection, and impaired production of normal immunoglobulin are common clinical manifestations of multiple myeloma. It is often also characterized by diffuse osteoporosis, usually in the pelvis, spine, ribs, and skull.

In multiple myeloma, a cancer cell forms a system of pathways and feedback loops to a bone marrow stromal cell with specific molecular agents in the pathways. This system is highly resilient and develops alternative pathways when under attack. The effects of these interactions are the exponential prolongation of the life of a cancer cell, defeating apoptosis or natural cell death, accelerated myeloma cell proliferation, and production of M protein. A single element therapy, for example chemotherapy administered at high doses, cannot compete with this cancer dynamic. Associated toxicity and/or the cancer's ability to use alternative pathways or to develop new adaptive mechanisms will always limit or avoid the effect of a single element therapy.

It was known in the art to use thalidomide, which was believed to interfere with the generation of blood vessels, and dexamethasone, which contains the proliferation of myeloma cells, can be used in combination to treat multiple myeloma. It was also known that it is necessary to minimize thalidomide and dexamethasone to reduce side effects. Further, it was known in the art that the Reishi mushroom, a traditional Chinese medicine, has a salubrious effect on the immune system (see U.S. Pat. No. 6,630,160).

In accordance with one aspect of the invention, potentiation was obtained from three components of the invention primary therapy elements, secondary therapy elements, and a computer program therapy analyzer. Specifically, primary therapy elements included thalidomide, dexamethasone, and Reishi (ganoderma lucidum). Secondary therapy elements included nutritional supplements such as magnesium aspartate, potassium gluconate, ferrous gluconate, vitamin B complex, controlled exposure to natural sunlight (facilitating production of vitamin A in cats) and EPA/DHA (highly unsaturated fats in fish oil), administration, and dose. Correlation factors included appetite, waking weight, metabolism, balance/equilibrium, mental acumen, physical strength or endurance, dexterity, attitude, pain, blood values, vital signs, and side effects, e.g. peripheral neuropathy (manifested by shaking of paws) and constipation. The method of the invention analyzed correlation between a selected therapy element and a correlation factor. Monitoring food intake is non-invasive and was chosen as the correlation factor for the primary element, dexamethasone. Other elements were monitored on a daily basis or less frequently. For example, serum calcium was measured approximately every three months.

The following table is exemplary of the recorded pairs of therapy elements and correlation factors employed in this study:

| | | Administration | Correlation Factors |
|---|---|---|---|
| | Dose (D) | Frequency | (CF) |
| Primary Therapy | | | |
| Elements (P) | | | |
| Pa - Thalidomide | 1.9 mg/day | Oral, Compounded; | CFa - Peripheral |
| | | .95 ml, | Neuropathy - Dose |
| | | 9:30 PM Daily | Setting Factor |
| Pb - Thalidomide | 1.9 mg/day | Oral, Compounded | CFb - Constipation |
| (continued) | | | |
| Pc - Thalidomide | 1.9 mg/day | Oral, Compounded | CFc - Serum |
| (continued) | | | Calcium |
| Pd - Thalidomide | 1.9 mg/day | Oral, Compounded | CFd - M Protein |
| (continued) | | | |
| Pe - Thalidomide | 1.9 mg/day | Oral, Compounded | CFe - Plasma Cells |
| (continued) | | | in Bone Marrow |
| Pf - Dexamethasone | .175 mg Modulated | Oral, Compounded; | CFf - Food Intake - |
| | (2 days on, 1 day | .7 ml | Dose Setting Factor |
| | off) | | |
| Pg - Dexamethasone | .175 mg Modulated | Oral, Compounded | CFc - Serum |
| (continued) | (2 days on, 1 day | | Calcium |
| | off) | | |
| Pg - Dexamethasone | .175 mg Modulated | Oral, Compounded | CFd - M Protein |
| (continued) | (2 days on, 1 day | | |
| | off) | | |
| Pg - Dexamethasone | .175 mg Modulated | Oral, Compounded | CFe - Plasma Cells |
| (continued) | (2 days on, 1 day | | in Bone Marrow |
| | off) | | |
| Pg - Dexamethasone | .175 mg Modulated | Oral, Compounded | CFg - GI Distress |
| (continued) | (2 days on, 1 day | | |
| | off) | | |
| Ph - Dexamethasone | .175 mg Modulated | Oral, Compounded | CFh - Serum |
| (continued) | (2 days on, 1 day | | Potassium Loss |
| Pi - Reishi | 80 mg/day | Oral, Compounded; | CFi - MM |
| | | .8 ml | Responsiveness to |
| | | | Dexamethasone |
| Pj - Reishi (continued) | 80 mg/day | Oral, Compounded | CFj - GI Distress |
| Pj - Reishi (continued) | 80 mg/day | Oral, Compounded | CFc - Serum |
| | | | Calcium |
| Pj - Reishi (continued) | 80 mg/day | Oral, Compounded | CFd - M Protein |
| Pj - Reishi (continued) | 80 mg/day | Oral, Compounded | CFe - Plasma Cells |
| | | | in Bone Marrow |
| Secondary Therapy | | | |
| Elements (S) | | | |
| Sk - Magnesium | 37.5 mg/day | Oral, Compounded | CFk - Reduced |
| Aspartate | | | Peripheral |
| | | | Neuropathy |
| Sl - Adrenal | 25 mg/day | Oral, Compounded | CFl - Reduction in |
| Supplement | | | the Required |
| | | | Amount of |
| | | | Dexamethasone |
| Sm - Ferrous | 9 mg/day | Oral, Compounded | CFm - RBC |
| Gluconate | | | (Anemia) |
| Sn - EPA/DHA | 54 mg/36 mg/day | Oral, Compounded | No Specific |
| | | | Correlation Factor |
| So - Potassium | 60 mg/day | Oral, Compounded | CFo - Serum |
| Gluconate | | | Potassium Level |
| Sp - B Complex | 19.6 mg, 1 day per | Oral, Compounded | No Specific |
| | week | | Correlation Factor |

The inventive method was used to analyze changes from day to day. The data was digitized by substituting either +1 for positive change (dayₙ₊ᵢ greater than dayₙ), 0 (zero) for no change, and −1 for negative change (dayₙ₊ᵢ less than dayₙ). Digitizing the change magnifies small changes by giving small differences the same value as large differences. For example, a positive change is noted as +1 whether the change is 1 milligram or 10 grams.

The inventive method multiplied a digitized change in therapy element by the corresponding digitized change in correlation factor to produce a correlation product. Because the digitized changes are signed (+ or −), changes in the same direction (both positive or both negative) produce a positive product. Changes in opposite directions (one negative and one positive) produce a negative product. The inventive method then provided a rolling average of correlation products over fixed period of time. (A positive average indicates a positive correlation between dose and result.)

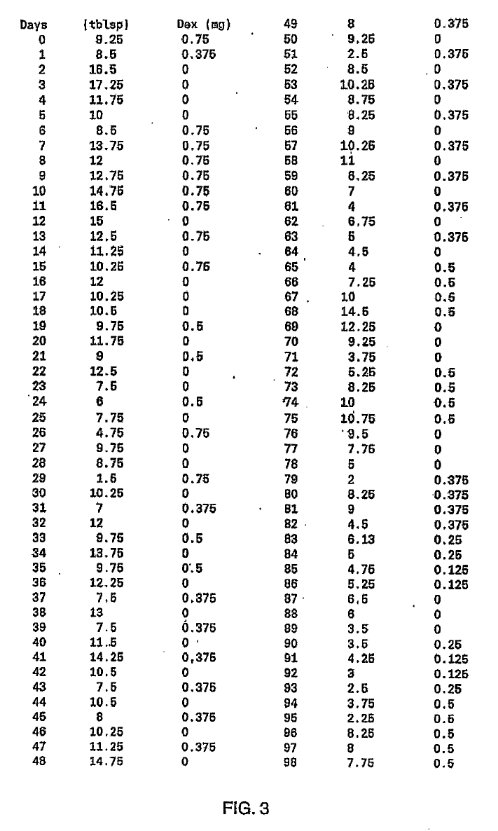

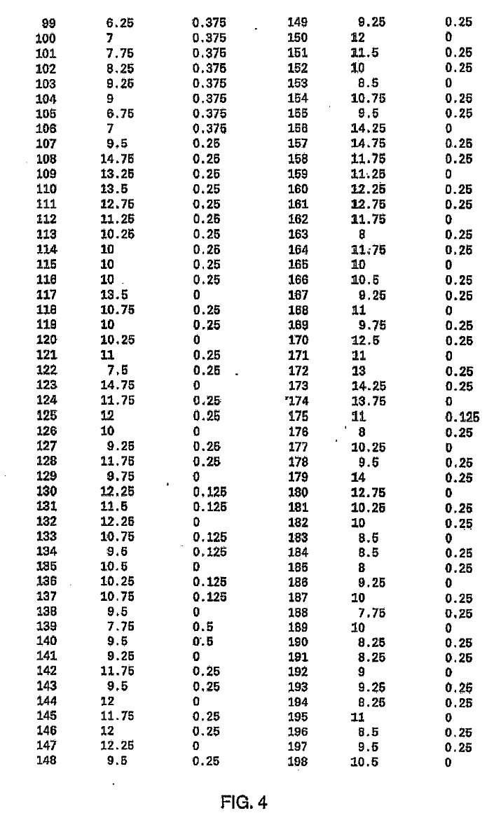

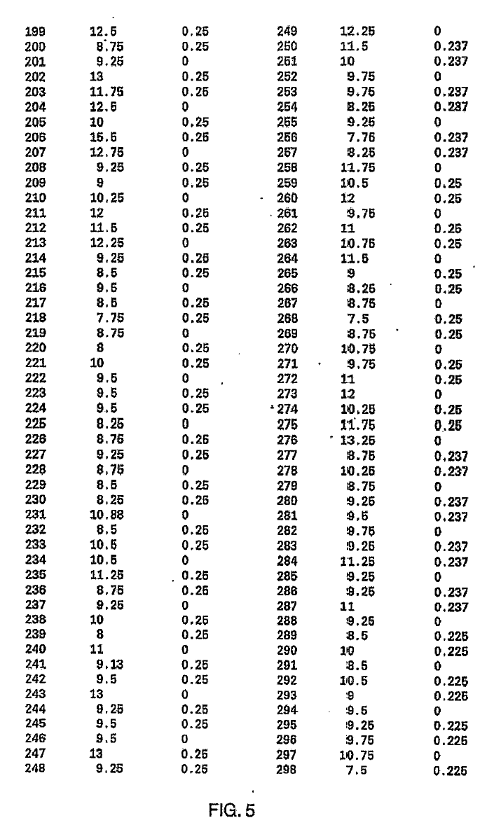

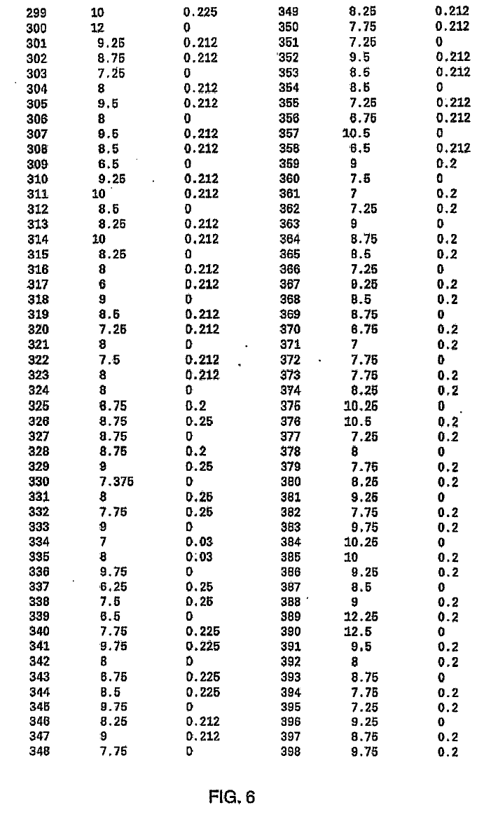

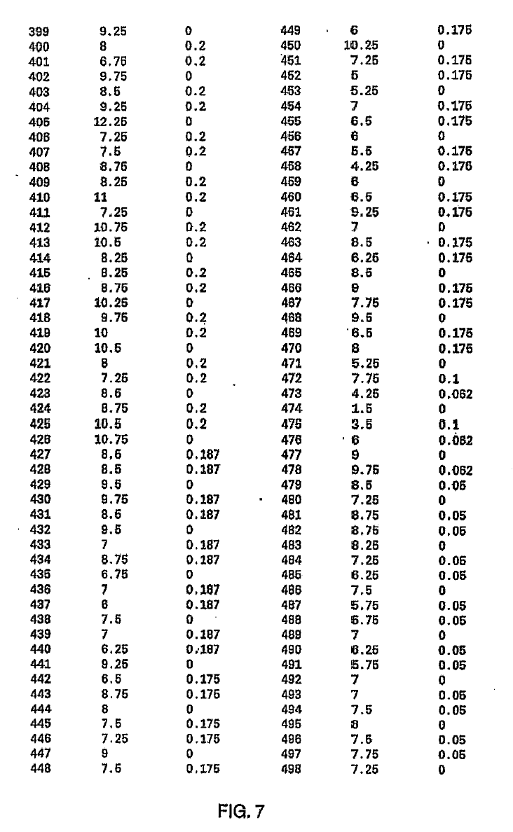

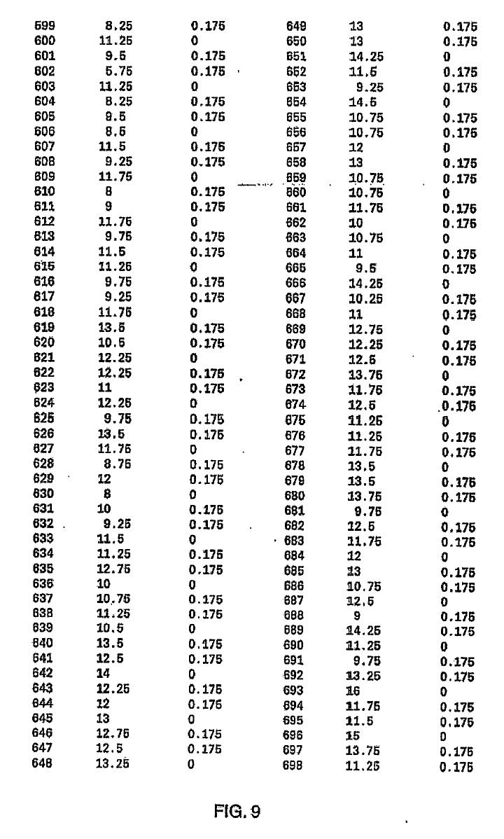

FIG. 3 through FIG. 10 are tables of data collected. (Unless otherwise indicated, these tables contain the data used for producing other figures in the drawings.) All manipulation of the data was readily done using a spreadsheet program.

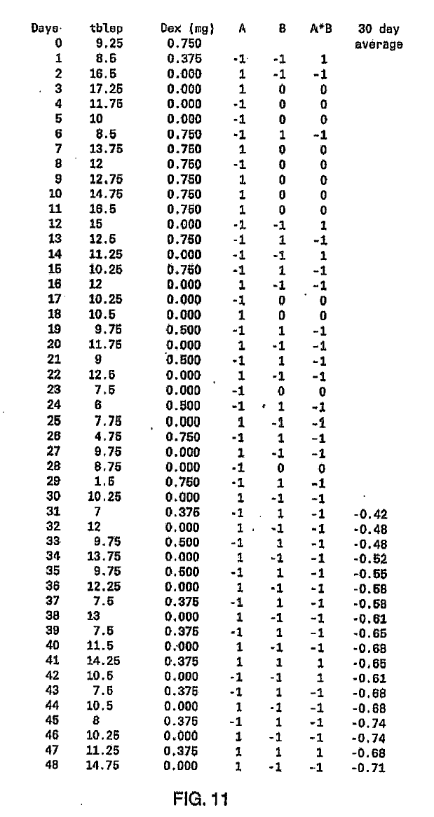

FIG. 11 illustrates the operation of an exemplary computer program product in accordance with an aspect of the invention. Column A is the change in food intake from day, to dayₙ₊ᵢ. Food intake decreased from day 0 (zero) to day 1 and the first entry in column A is therefore −1. Column B is the change in the amount of dexamethasone given from day, to dayₙ₊ᵢ. Dosage decreased from day 0 to day 1 and the first entry in column B is therefore −1. Column A×B is the product of column A and column B. The first entry is +1, or 1.

The column labeled “30 day average” is a rolling average. As can be seen, initially, there appeared to be a negative correlation between dosage and food intake. In this embodiment, interpreting data is left to a clinician and an understanding of the medicines used is imperative. It was known that thalidomide takes time to show effect, from two weeks to eight months. Dexamethasone was also known to take time to show effect, approximately twenty four hours, and to have many side effects, including causing gastric distress, which affects food intake. Thus, generally, dexamethasone was not given every day, although there was an initial period, day 6 and following, when it was given every day to stabilize the patient.

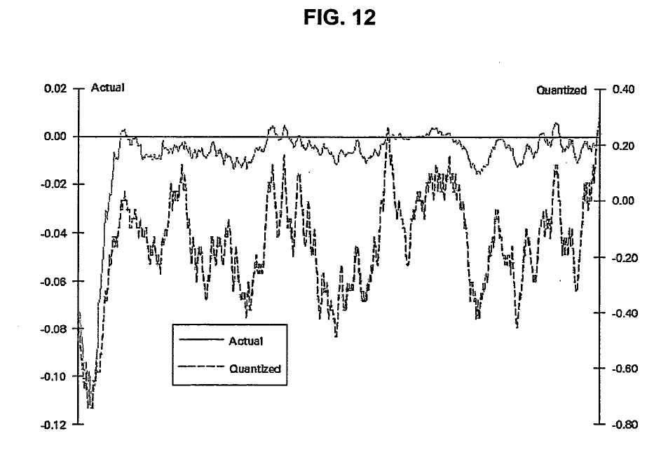

The computer program product also provided by the invention generated a graphical display of the data. FIG. 12 is a graph of the thirty day rolling average of the correlation products for all the data in FIGS. 3 to 10. The abscissa is days and the ordinate is average correlation product. The dashed line represents digitized (“quantized”) data. The solid line represents actual differences from day to day. Correlation appeared to be negative for either curve. An important feature of FIG. 12 is the averaging period. One wants a period long enough to smooth the data but not so long as to distort the information contained therein.

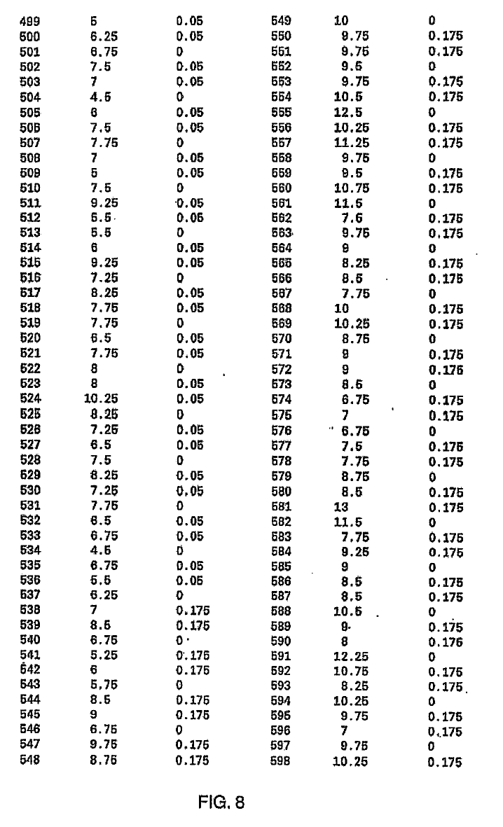

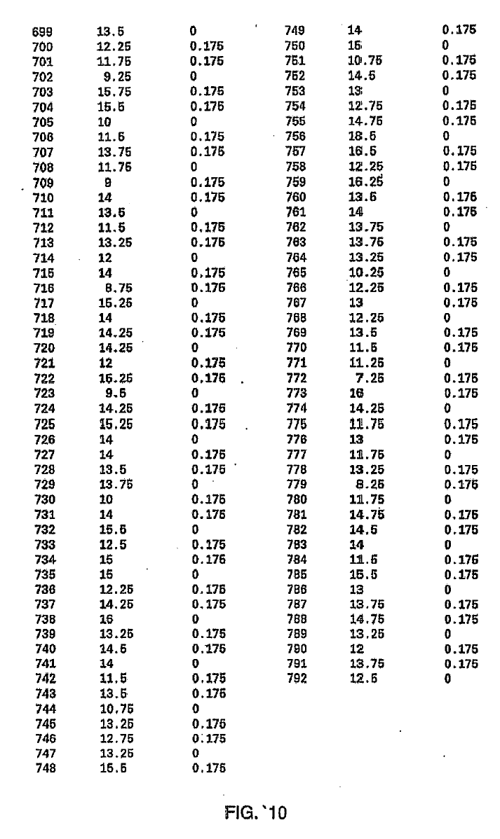

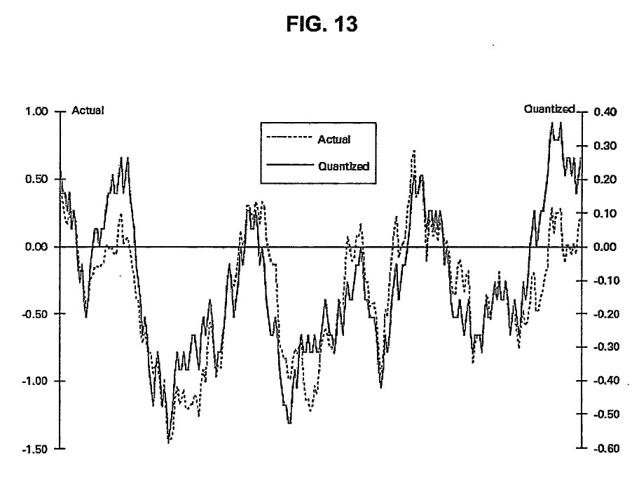

FIG. 13 depicts the data from day 537 (FIG. 8) through day 792 (FIG. 10) is used. During this period, the administration of dexamethasone was modulated at two days on followed by one day off and the dose was a constant 0.175 mg. In FIG. 13 the averaging period is reduced to eighteen days, an even multiple of the modulation period of three days. Again, digitized data and non-digitized data are presented. Because the dosage was either zero or 0.175 mg., the administration data is, in effect, digitized because the change in dose is either zero, +x, or −x, with x=0.175 rather than x=1 as in FIG. 11.

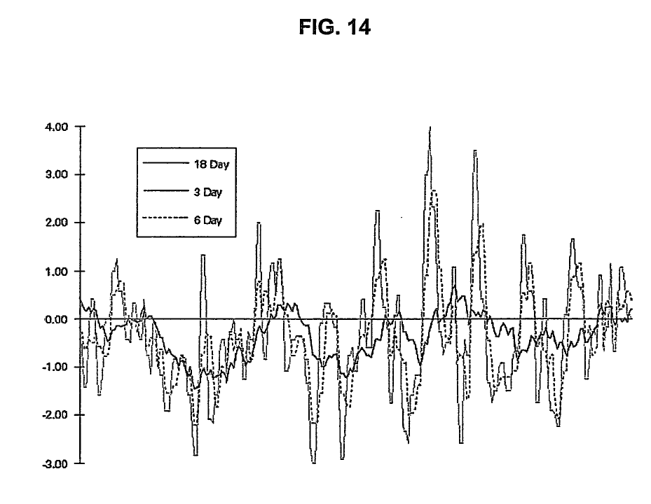

In FIG. 14, based on the same undigitized data as for FIG. 13, the averaging period is three, six, or eighteen days. An averaging period that is several times the modulation period provides smoother data. An averaging period that is at least four times the modulation period is preferred.

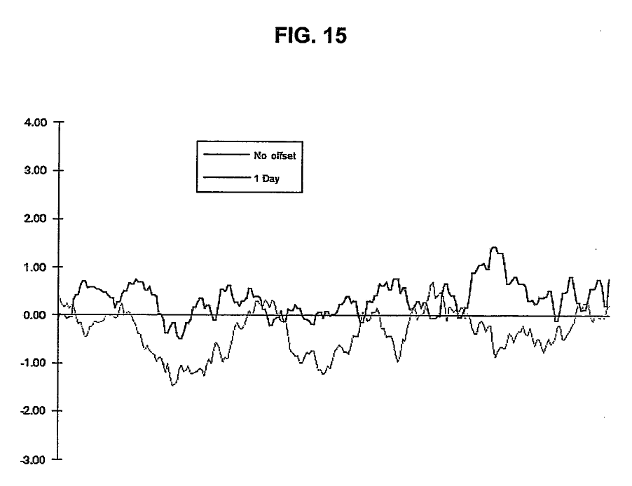

Cats have a delayed reaction to dexamethasone. On the day that dexamethasone is given, appetite is expected to be lower and this was observed. This has a negative effect on the average because dosage is increased (the previous day's dose was zero) and appetite is decreased. Thus, the changes are in opposite directions and the product is negative. There is a phase or timing relationship that should be considered. FIG. 15, drawn to the same scale as FIG. 14, illustrates an eighteen day rolling average of correlation product with a one day offset for the change in food intake. That is, the food intake on dayₙ₊ᵢ minus the food intake on dayₙ is multiplied by the dose on dayₙ minus the dose on dayₙ_{_{—}}ᵢ. In FIGS. 12-14, the product is the food intake on dayₙ minus the food intake on dayₙ_{_{—}}ᵢ multiplied by the dose on dayₙ minus the dose on dayₙ_{_{—}}ᵢ. As clear from FIG. 15, the offset radically changes the result, showing a strong positive correlation for the therapy.

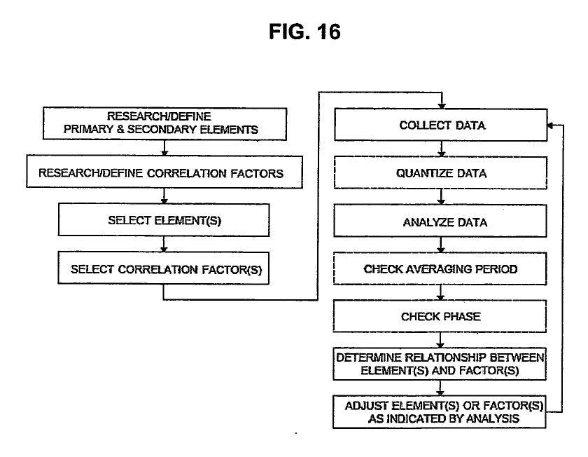

FIG. 16 is a flow chart illustrating the process for potentiating this cat's therapy. Many decisions precede the first step of the flow chart, such as deciding between types of therapy, e.g. debulking and immunomodulation; elemental, linear, or composite treatment. It is presumed that the potentiation process has been chosen.

The first step was to research primary elements, secondary elements, and correlation factors. From these, at least one primary element, or at least one secondary element, and at least one correlation factor was chosen. Relevant data was collected and, preferably, digitized. Such data included dose, administration, vital signs, and other parameters relating to the condition of the patient. The correlation products were calculated as described above and the average of the products displayed as illustrated in FIG. 12, FIG. 13, FIG. 14 or FIG. 15. Optionally, one checks averaging period and phase (e.g., time offset), as discussed above. Based upon the results of the display, the selection of elements and correlation factors or other treatments was adjusted or modified.

The cat's condition had made the transition from acute and critical to chronic, but manageable. The proper administration of dexamethasone was established. Thus, potentiating the therapy produced a stable patient with the possibility of inhibiting the cancer's ability to defeat apoptosis. The standard treatment for a cat is chemotherapy, using other drugs than the ones described above, having a reported remission rate of forty percent with a median survival period of one hundred seventy days.

Potentiating the cat's therapy also established a “narrow therapeutic band” for the patient with the following characteristics:

The lowest thalidomide dose administered was 1.6 mg. At thalidomide doses below 1.6 mg, there is a concern that therapeutic effect will drop off. However, this minimum dose, in combination with the other elements of the potentiating protocol, controlled the disease without significant thalidomide side effects or resistance.

At thalidomide doses above 1.8 mg, therapeutic effect was not increased while adverse side effects, such as peripheral neuropathy, increased.

The lowest dexamethasone dose administered has been 0.125 mg. At dexamethasone doses below 0.125 mg (modulated 2 days on, 1 day off), it appeared that therapeutic effect drops. However, this minimum dose, in combination with the other elements of the potentiating protocol, controlled disease without significant side effects.

At dexamethasone doses above 0.162 mg, therapeutic effect is constant, while adverse side effects, such as thromboses and peripheral neuropathy, increased.

### EXAMPLE 3

This Example demonstrates the utility of time offset data analysis. Because of the delayed effect of some therapies, the invention provides for an “offset,” or staggering, of the corresponding therapy elements and correlation factors that are multiplied to obtain the normalized Delta Correlation Function (DCF).

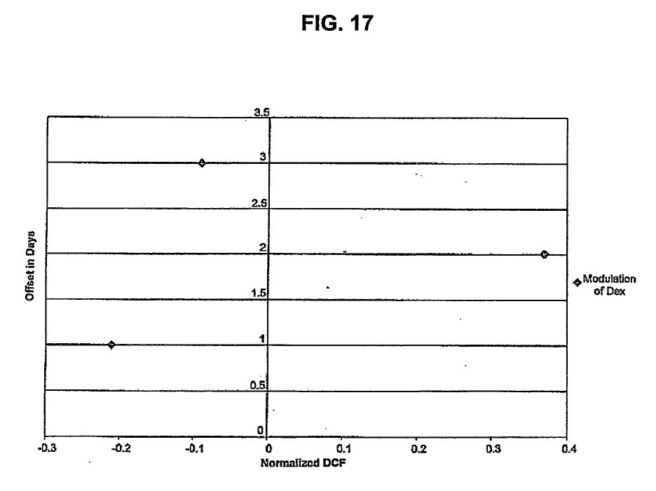

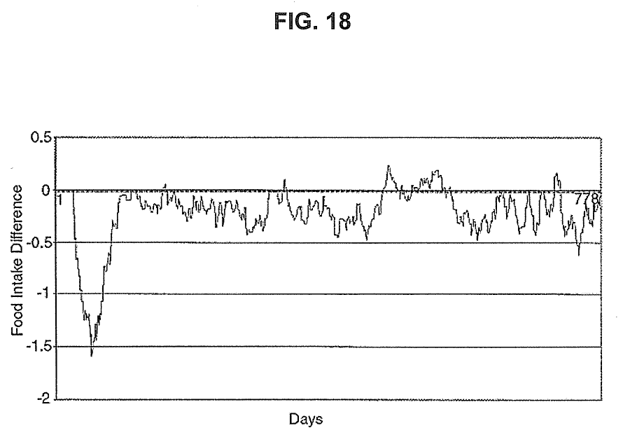

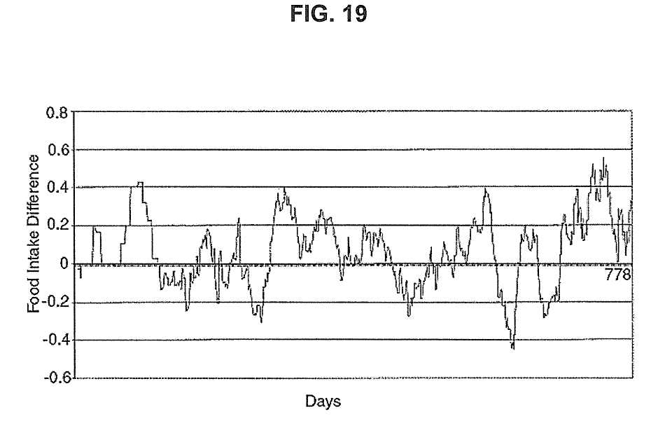

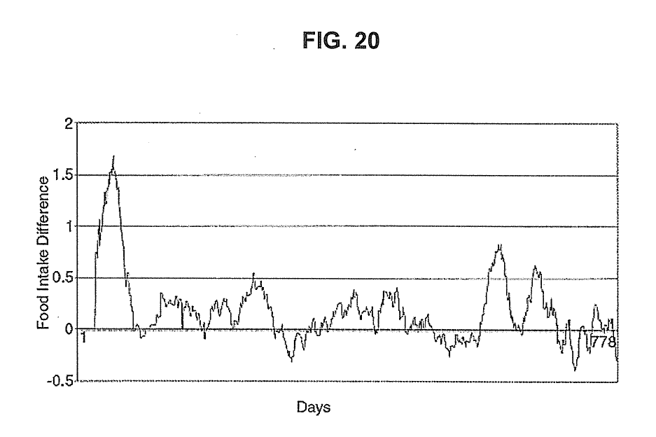

A method provided by the invention was used to optimize the dexamethasone regimen based on the correlation factor food intake (i.e., determined the dexamethasome regimen that produces an increase in food intake.) Again, the cat myeloma model was used. A regimen of 0.175 mg/day dexamethasone administered using a 2 days on, 1 day off schedule was evaluated. The normalized DCF was calculated based upon a 24 time point sample (Days 445 to 469) with various offsets. Correlating changes in food intake with the dexamethasone dose on a same day basis showed a −0.16 normalized DCF (a 16% probably that an increase in dexamethasone will produce a decrease in food intake). When the change in food intake is offset by 1 day, there was an even more negative effect on food intake with a −0.21 normalized DCF. However, when a 2 day offset was used the normalized DCF shows a strong positive effect on food intake with a +0.37 normalized DCF. A 3 day offset again the results again turn negative with a −0.09 normalized DCF (FIG. 17). The 30 Day rolling averages for food intake also show different patterns for no offset (FIG. 18), a 1 day (FIG. 19), and a 2 day offset (FIG. 20). Notably, a 1 day offset shows a recognizably more positive correlation pattern.

In another example, a 0.375 mg of dexamethasone was administered over Days 37 to 61 on a 1 day on, 1 day off basis. While the same day normalized DCF is −0.52 (indicating the regimen was very bad for the patient), with a 1 day offset, the normalized DCF becomes +0.5 (indicating the regimen was very good for the patient). These results are likely due to the time required for the dexamethasone therapeutic effect to develop, approximately 1 day in this regimen.

Based on the foregoing analysis, 3 different dexamethasone regimens where designed and hypothesized systems functions were formulated for predicting the impact of a regimen on food intake. Most significantly, the recognition of an offset pattern allowed for the use of linear regression analysis of the data collected from a 130 day timecourse to generate hypothetical systems functions which incorporated the dose and dosing cycle. In general, the system functions are in the form of:

Predicted Food Intake Dayₙ (tsp)=(dexamethasone dose Dayₙ) (mg)×Factor Dayₙ (tsp/mg);

Predicted Food Intake Dayₙ₊₁ (tsp)=[(dexamethasone dose Dayₙ₊₁) (mg)×Factor Dayₙ₊₁ (tsp/mg)]+Predicted Food Intake Dayₙ;

Predicted Food Intake Dayₙ₊₂ (tsp)=[(dexamethasone dose Dayₙ₊₂) (mg)×Factor Dayₙ₊₂ (tsp/mg)]+Predicted Food Intake Dayₙ₊₁;

wherein “Dayₙ” is the first day of a three to six day dosing cycle and, if the dexamethasone dose is 0.00, this value is converted to 1.00. “Factor Dayₙ” is regimen specific and was determined by linear regression analysis of the correlation between dexamethasone dose and food intake performed separately for days 1, 2, and 3 of the cycle a given regimen. For example, all first day dexamethasone administrations were identified in the past data. Food intake for those days were compared to the food intake the day before and averaged, then divided by the dose of dexamethasone for each first day (((Food Intake Dayn (tsp)−Dayₙ₋₁ (tsp))/number of first days)/dexamethasone Dayn).

The system functions were used to predict the results with three dexarnethasone regimens. Regimen 1 employs a dosing schedule of 0.250 mg dexamethasone 2 days on and then 1 day off. The System Function postulated for Regimen 1 is:

Predicted Food Intake Dayₙ (tsp)=0.250 (mg)×−5.11 (tsp/mg);

Predicted Food Intake Dayₙ₊₁ (tsp)=[0.250 (mg)×8.57 (tsp/mg)]+Predicted Food Intake Dayₙ; and

Predicted Food Intake Dayₙ₊₂ (tsp)=[(1.00 (mg)×2.23 (tsp/mg)]+Predicted Food Intake Dayₙ₊₁.

Regimen 2 employs a dosing schedule of 0.250 mg dexamethasone 2 days on and then 1 day at 0.125 mg dexamethasone. The system function postulated for Regimen 2 is:

Predicted Food Intake Dayₙ (tsp)=0.250 (mg)×−5.11 (tsp/mg);

Predicted Food Intake Dayₙ₊₁ (tsp)=[0.250 (mg)×8.57 (tsp/mg)]+Predicted Food Intake Dayₙ; and

Predicted Food Intake Dayₙ₊₂ (tsp)=[0.125 (mg)×−0.53 (tsp/mg)]+Predicted Food Intake Dayₙ₊₁.

Regimen 3 employs a dosing schedule of 0.125 mg dexamethasone every day. The System Function postulated for Regimen 3 uses a six day cycle:

Predicted Food Intake Dayₙ (tsp)=0.125 (mg)×−5.11 (tsp/mg);

Predicted Food Intake Dayₙ₊₁ (tsp)=[0.125 (mg)×8.57 (tsp/mg)]+Predicted Food Intake Dayₙ;

Predicted Food Intake Dayₙ₊₂ (tsp)=[0.125 (mg)×−0.53 (tsp/mg)]+Predicted Food Intake Dayₙ₊₁;

Predicted Food Intake Dayₙ₊₃ (tsp)=[0.125 (mg)×−0.10 (tsp/mg)]+Predicted Food Intake Dayₙ₊₂;

Predicted Food Intake Dayₙ₊₄ (tsp)=[0.125 (mg)×3.89 (tsp/mg)]+Predicted Food Intake Dayₙ₊₃; and

Predicted Food Intake Dayₙ₊₅ (tsp)=[0.125 (mg)×−1.72 (tsp/mg)]+Predicted Food Intake Dayₙ₊₄.

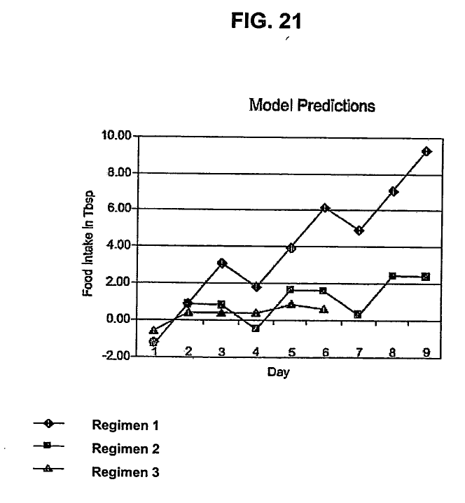

As shown in FIG. 21 Regimen 1 is predicted to yield the greatest improvements in food intake. This proved to be the case, with the use of the Regimen 1 regimen food intake by the test animal reached the physiologic limit of 15.5 tsp per day. Thus, time offset analysis, as provided by the invention, can lead to the formulation of System Functions, which in turn can lead to optimized therapy.

From the forgoing it can be seen that the invention provides methods for analyzing data to correlate treatment and symptoms and accommodate changes in treatment with changes in the condition of the patient so as to determine the minimum dose for maximum therapeutic effect, minimal side effects, and improved patient stability.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for potentiating a therapy in a patient comprising the steps of:
(a) selecting a plurality of therapy elements;
(b) selecting a plurality of correlation factors;
(c) collecting data relative to the therapy elements and the correlation factors at a plurality of time points in a timecourse; and
(d) analyzing the data by
(i) calculating a difference between the therapy elements for each successive pair of time points and a difference between the correlation factors for each successive pair of time points;
(ii) processing the difference by applying a function from the group consisting of digitizing each of the calculated differences and normalizing each of the calculated differences;
(iii) calculating a product of the processed difference for each therapy element from each successive pair of time points multiplied by the corresponding digitized or normalized difference for each correlation factor from each successive pair of time points;
(iv) calculating respective rolling averages of the products over the timecourse;
(v) displaying one or more of the rolling averages; and
(vi) modifying the therapy in accordance with the display.

2. The method of claim 1, wherein digitized differences are used in step (iii).

3. The method of claim 1, wherein normalized differences are used in step (iii) and the calculated differences are normalized by calculating the percent change the differences represent.

4. The method of claim 1, wherein the method is repeated one or more times modifying one or more therapy elements on each repetition of the method.

5. The method of claim 4, wherein the method is repeated until at least one predetermined outcome metric is satisfied.

6. The method of claim 1, wherein a data reduction step is inserted between step (iv) calculating a rolling average of the products over all of the timecourse and step (v) displaying the rolling averages, and wherein the data reduced rolling averages are displayed.

7. The method of claim 6, wherein the inserted data reduction step comprises the application of a protocol selected from the group consisting of cluster analysis, principle component analysis, factor analysis, discriminant function analysis, multidimensional scaling, and combinations thereof.

8. The method of claim 6, wherein the data reduction protocol identifies the therapy elements producing the greatest variation in the data, the therapy elements producing the greatest variation in the data are displayed, and therapy is modified by modifying one or more of the identified therapy elements producing the greatest variation in the data in accordance with the display.

9. The method of claim 8, wherein the modifications are based on modification rules.

10. The method of claim 9, wherein the method is repeated until at least one predetermined outcome metric is satisfied.

11. The method of claim 10, wherein at least one outcome metric has at least one of the properties of measuring a physiologic parameter, measuring patient performance status, measuring patient symptom, measuring patient reported subjective factor, measuring family reported subjective factor, measuring side effects, measuring complications, measuring cost or measuring compliance with a narrow therapeutic band.

12. The method of claim 1, wherein the therapy is directed at a disease from the group consisting of neoplastic diseases, infectious diseases, autoimmune diseases, metabolic diseases, psychiatric diseases, and combinations thereof.

13. The method of claim 12, wherein the disease is selected from the group consisting of hyperplasias, benign tumors, atypical hyperplasias, carcinomas in situ, carcinomas, sarcomas, teratomas, lymphomas, leukemias, and plasma cell dyscarsias, viral infections, bacterial infections, fungal infections, polymicrobial infections, lupus, arthritis, mixed connective tissue disease, inflammatory bowel disease, graph versus host disease, transplant rejection, diabetes, metabolic syndrome, hyperlipidemia, psychoses, anxiety disorders, affective disorders, attention disorders, and personality disorders.

14. The method of claim 1, wherein the therapy is directed at an acute condition.

15. The method of claim 14, wherein the acute condition is selected from the group consisting of birth, sepsis, stroke, myocardial ischemia, septic shock, shock trauma, surgery, and combinations thereof.

16. The method of claim 1, wherein at least one therapy element is selected from the group consisting of chemotherapeutic agents, radiation therapies, surgical procedures, antibiotics, antifungals, antivirals, hormones, immuno-modulators, vaccines, NSAIDs, opiates, analgesics, anaesthetics, antihypertensives, anticoagulants, oral hypoglycemics, statins, fibrates, neuroleptic, antidepressant, lithium, anxiolytic, herbal extracts, traditional medicines, psychotherapy, nutritional supplement, diet, and combinations thereof.

17. The method of claim 1, wherein at least one correlation factor is selected from the group consisting of physiologic parameters, patient performance status, patient symptoms, patient reported subjective factors, family reported subjective factors, complications, costs, lawsuits, and combinations thereof.

18. A computer program product comprising a computer-readable medium having thereon computer-executable instructions for the performance of a method for potentiating a therapy in a patient, said method comprising the steps of:
(a) selecting a plurality of therapy elements;
(b) selecting a plurality of correlation factors;
(c) collecting data relative to the therapy elements and the correlation factors at a plurality of time points in a timecourse;
(d) storing the collected data in an relational database; and
(e) analyzing the data by:
(i) calculating a difference between the therapy elements for each successive pair of time points and a difference between the correlation factors for each successive pair of time points;
(ii) processing the therapy element differences and correlation factor differences by applying a function selected from the group consisting of digitizing each of the calculated differences and normalizing each of the calculated differences;
(iii) calculating the product of the digitized or normalized difference for each therapy element for each successive pair of time points multiplied by the digitalized or normalized difference for each correlation factor for each successive pair of time points;
(iv) calculating a rolling average of the products over the timecourse;
(v) displaying one or more of the rolling averages; and
(vi) providing a recommendation for a modification of the therapy in accordance with the display.

19. The computer program product of claim 18, wherein a data reduction step is inserted between step (iv), calculating a rolling average of the products over all of the time points, and step (v), displaying the rolling averages, and wherein the data reduced rolling averages are displayed.

20. The computer program product of claim 19, wherein the data reduction step is a protocol selected from the group consisting of cluster analysis, principle component analysis, factor analysis, discriminant function analysis, multidimensional scaling, and combinations thereof.

21. The computer program product of claim 19, wherein the data reduction protocol identifies the therapy elements producing the greatest variation in the data and, if one or more outcome metrics are not satisfied, the recommendation for a modification of the therapy elements recommends the modification of one or more of the therapy elements producing the greatest variation in the data.

22. The computer program product of claim 18, wherein the therapy is directed at a disease selected from the group consisting of hyperplasias, benign tumors, atypical hyperplasias, carcinomas in situ, carcinomas, sarcomas, teratomas, lymphomas, leukemias, and plasma cell dyscarsias, viral infections, bacterial infections, fungal infections, polymicrobial infections, lupus, arthritis, mixed connective tissue disease, inflammatory bowel disease, graph versus host disease, transplant rejection, diabetes, metabolic syndrome, hyperlipidemia, psychoses, anxiety disorders, affective disorders, attention disorders, and personality disorders.

23. The computer program product of claim 18, wherein the therapy is directed at a condition selected from the group consisting of birth, sepsis, stroke, myocardial ischemia, septic shock, shock trauma, surgery, and combinations thereof.

24. The method of claim 18, wherein the recommendation is step (e) (vi) is based upon the calculated products of the processed difference for each therapy element for each successive pair of time points multiplied by the processed difference for successive pairs of correlation factor time points, wherein each correlation factor pair of time points is offset in time by a fixed interval from each therapy element pair.

25. A method for potentiating a therapy in a patient comprising the steps of:
(a) selecting a plurality of therapy elements;
(b) selecting a plurality of correlation factors;
(c) collecting data relative to the therapy elements and the correlation factors at a plurality of time points in a timecourse;
(d) analyzing the data by
(i) calculating a difference between the therapy elements for each successive pair of time points and a difference between the correlation factors for each successive pair of time points;
(ii) processing the difference by applying a function from the group consisting of digitizing each of the calculated differences and normalizing each of the calculated differences;
(iii) calculating a product of the processed difference for each therapy element from each successive pair of time points multiplied by the corresponding digitized or normalized difference for each correlation factor from each successive pair of time points;
(iv) calculating respective rolling averages of the products over the timecourse;
(v) using linear regression analysis to determine a System Function for each therapy element-correlation factor pair;
(e) displaying one or more of the rolling averages and one or more of the Systems Functions; and
(f) modifying the therapy in accordance with the displayed rolling averages and Systems Functions
